(19) United States
(12) Patent Application Publication
(10) Pub. No.: US 2023/0045009 A1
(43) Pub. Date: Feb. 9, 2023

(54) TREATMENT OF THE CORNEA USING CROSSLINKING AND MECHANICAL LOAD

(71) Applicant: THE TRUSTEES OF COLUMBIA UNIVERSITY IN THE CITY OF NEW YORK, New York, NY (US)

(72) Inventor: SINISA VUKELIC, NEW YORK, NY (US)

(21) Appl. No.: 17/890,905

(22) Filed: Aug. 18, 2022

Related U.S. Application Data
(63) Continuation of application No. PCT/US2021/018626, filed on Feb. 18, 2021.
(60) Provisional application No. 63/044,928, filed on Jun. 26, 2020.
(60) Provisional application No. 62/991,501, filed on Mar. 18, 2020.
(60) Provisional application No. 62/978,086, filed on Feb. 18, 2020.

(51) Int. Cl. A61F 9/008

(52) U.S. Cl. CPC A61F 9/008; A61F 2009/00872; A61F 2009/00893; A61F 2009/00842

(57) ABSTRACT

A method of treatment of the cornea of an eye including exposing the cornea to a crosslinking medium, and applying a mechanical loading to the cornea, wherein the mechanical loading is selected as a strain proportional to the dimensions of the eye being treated. A method of altering the curvature of the cornea is provided including controlling a light source to apply light energy pulses to corneal tissue; wherein the light energy pulses are below an optical breakdown threshold for the cornea; ionize water molecules within the treated corneal layer to generate reactive oxygen species; and initiate crosslinking within the extracellular matrix of the cornea to change the physical properties of the cornea, e.g., the stiffness of the cornea.

### CROSS-REFERENCE TO RELATED APPLICATIONS

This application is continuation of PCT/US21/18626 entitled “Treatment of the Cornea Using Crosslinking and Mechanical Load”, filed Feb. 18, 2021, which claims priority to U.S. Provisional Application No. 62/978,086, filed Feb. 18, 2020, entitled “Treatment of the Cornea Using Crosslinking and Mechanical Load”; U.S. Provisional Application No. 62/991,501, filed Mar. 18, 2020, entitled “Treatment of the Cornea Using Crosslinking Field”; and U.S. Provisional Application No. 63/044,928, filed Jun. 26, 2020, entitled “Femtosecond Laser Crosslinking As A Platform For Various Treatment Modalities,” which are incorporated by reference in their entirety herein.

### FIELD

The present disclosure relates to techniques for treatment of the cornea using crosslinking and mechanical loading.

### SUMMARY

Keratoconus (KCN) is a condition in which cornea assumes conical shape due to noninflammatory thinning of corneal stroma. The condition usually begins in adolescence and progresses in next two decades, resulting in irregular astigmatism, myopia and protrusion. Severity and the rate of progression is variable, and ranges from mild astigmatism to severe corneal thinning, and scarring. In advanced KCN cases rupture in Descement's membrane is reported, which results in acute corneal hydrops. Although there were some connections with other diseases, KCN is mostly isolated condition. 10-20% of KCN patients eventually undergo penetrating keratoplasty, with KCN being the most common reason for keratoplasty in the developed world.

Etiology of KCN is yet to be fully understood, however it is implicated that KCN has a multifocal origin with biochemical, physical, and genetic pathways implicated. KCN can occur due to genetic predisposition, while triggered by environmental factors. About 10% of cases are have been linked with family history and some correlations have been established between KCN and rare genetic disorders such as Leber's congenital amaurosis and Down syndrome. KCN is also associated with atopy, hard contact lens wearing and eye rubbing. The current consensus revolves around interaction between environmental and endogenous factors.

Although exact pathways are unknown, it is assumed multitude of molecular events act in concert to produce KCN in eyes. KCN corneas have increased interleukin-1 (IL-1) expression, which is paired with elevated expression of IL-1 receptors in keratoconic fibroblasts. Inflammation and injury to corneal epithelium triggers secretion of IL-1, which together with other cytokines such as tumor necrosis factor-a (TNF-a) and interleukin-17 (IL-17), induces keratocyte apoptosis in the anterior stroma. In normal homeostasis, IL-1 regulates tissue organization through apoptotic and negative chemotactic effects on stromal keratocytes. However, in KCN eyes it likely leads to gradual loss of keratocytes and, over a period of time, reduction in stromal mass. Further, KCN corneas exhibit inhibitor-enzyme imbalance, such as matrix metalloproteinases (MMPs) and its inhibitors. a1-proteinase inhibitor and a2-macro-globulin are also found to have decreased levels in KCN corneas. Such an imbalance is likely to cause increased activity of degradative enzymes including trypsin, chymotrypsin, collagenase and elastase, leading to oxidative damage. The accumulation of damage alters signaling pathways, increases enzyme activity, induces keratocyte apoptosis, and fibrosis in KCN eyes. Environmental factors, such as mechanical trauma from excessive eye rubbing, sustained contact lens wear, and moderate to severe atopy likely promotes secretion of soluble immune mediators that bind to receptors in stromal keratocytes. Dysfunctional levels of proteinases, epithelial proteins and immunoglobulins result in keratocyte oxidative damage, which leads to apoptosis, keratocyte migration into the corneal epithelium, and differentiation of keratocytes into a-SMA expressing myofibroblasts. The result is tissue remodeling and production of KCN corneal architecture.

Management of KCN usually begins with spectacle correction, which is followed by prescription of specially fitted contact lenses to reduce KCN induced distortion. Keratoplasty is recommended if continued progression result in unacceptable vision. However, in the last decade corneal crosslinking gained traction as a promising treatment for KCN. De-epithelized eyes are soaked with a photosensitizer, usually riboflavin, and then exposed to ultraviolet-A (UVA) light. Crosslinking increases corneal rigidity and thus retards KCN progression. However, due to cytotoxicity, the treatment is considered safe only for sufficiently thick corneas. The restriction is unfortunate since corneal thinning is one of staples of KCN.

Corneal crosslinking (CxL) originated from the premise that UVA light can CxL tissues in presence of a photosensitizing agent. The idea was transplanted from polymer industry, and bioengineering. In late 1990s a group from Dresden suggested that strengthening cornea with CxL can have therapeutic effect, especially for keratoconus and other ectasias. It has since become a mainstream clinical practice.

Photochemical process is believed to be responsible for UVA/riboflavin CxL of corneal stroma. Riboflavin is excited with UVA light into singlet and triplet states, which enables production of reactive oxygen species (ROS). Oxygen availability governs ROS producing mechanisms. At low oxygen concentrations (Type I) excited photosensitizer produces radicals or radical ions, via hydrogen atom or electron transfer. In contrast, in aerobic conditions photooxidation of collagen fibrils occurs via interaction with a singlet oxygen (102), which is generated through reaction of excited riboflavin with the surrounding oxygen. In a typical CxL, Type II mechanism dominates CxL of the collagen matrix in first 15 seconds, after which oxygen is depleted and photoreaction continues under Type I mechanism. The procedure is also highly dependent of riboflavin penetration into the corneal stroma, which is why epithelial debridement is required.

### SUMMARY

In one aspect of the disclosed subject matter, a method of treatment of the cornea of an eye is provided including exposing the cornea to a crosslinking medium, and simultaneously applying a mechanical loading to the cornea, wherein the mechanical loading is selected as a strain proportional to a dimension of the eye.

In some embodiments, the mechanical loading is a 15% deformation (measured by a micrometer) of the original eye ball height. In some embodiment, the mechanical loading is provided by a 1.6 mm diameter cylinder stick.

In some embodiments, the crosslinking medium comprises at least one of UV-A light and riboflavin. In some embodiment, the mechanical loading is selected as a portion of the cornea.

In another aspect of the disclosed subject matter, a method of treatment of the cornea of an eye is provided including applying mechanical loading to the cornea; and irradiating a central region of the cornea with a femtosecond laser while the cornea is deformed by the mechanical loading.

In some embodiments, the mechanical loading is achieved by pressing the cornea with a cover slip.

In a further aspect of the disclosed subject matter, a method of treatment of the cornea of an eye is provided including applying mechanical loading to the cornea; and irradiating a central region of the cornea with a steepening technique with a femtosecond laser while the cornea is deformed by the mechanical loading, wherein the laser irradiation is applied in an annulus shaped pattern, such that apical region of cornea remains intact while the peripheral region is irradiated with the laser.

In a further aspect of the disclosed subject matter, a method of altering the curvature of the cornea is provided including controlling a light source to apply light energy pulses to corneal tissue; wherein the light energy pulses are below an optical breakdown threshold for the cornea; ionize water molecules within the treated corneal layer to generate reactive oxygen species; and initiate crosslinking within the extracellular matrix of the cornea to change the physical properties of the cornea, e.g., the stiffness of the cornea.

In some embodiments, the light source is a laser, e.g., a femtosecond laser. In some embodiments, the light energy pulses have an average power output between about 10 mW and about 100 mW, e.g., 60 mW. In some embodiments, the light energy pulses have a pulse energy between about 0.9 nJ and about 1.5 nJ, e.g., 1.2 nJ. In some embodiments, the light energy pulses have a wavelength of about 1060 nm.

In some embodiments, controlling the light source comprising applying the light energy pulses in one or more layers to the tissue. In some embodiments, the one or more layers are spaced about 50 microns apart. In some embodiments, controlling the light source comprising applying the light energy pulses in two to five layers to the tissue

In some embodiments, the method further including applying a mechanical loading to the cornea. In some embodiments, the mechanical loading is selected as a strain proportional to a dimension of the eye.

In further aspect of the disclosed subject matter, a method of treatment of the cornea of an eye is provided including exposing the cornea to a crosslinking medium, comprising application of a femtosecond oscillator, such that ionization of target molecules within cornea is achieved while avoiding damaging optical breakdown of the tissue.

In some embodiments, the method further includes applying a mechanical loading to the cornea. In some embodiments, the mechanical loading is selected as a strain proportional to a dimension of the eye.

### BRIEF DESCRIPTION OF THE DRAWINGS

FIG. 1 depicts the averaged difference, before treatment and after 12 hours, of a cornea subject to mechanical loading only when compared with simultaneous application of mechanical loading and crosslinking.

FIG. 2 depicts representative time-histories of effective refractive power variations over 12 hours for a pair for corneas subject to mechanical loading only when compared with simultaneous application of mechanical loading and crosslinking.

FIGS. 3A-3C depict representative refractive power maps from topographic measurements using exemplary embodiments of the disclosed subject matter.

FIGS. 3D-3F depict representative refractive power maps from topographic measurements using a control.

FIGS. 4A-4C depict representative OCT scans at apex cross section using exemplary embodiments of the disclosed subject matter.

FIGS. 4D-4F depict representative OCT scans at apex cross section using the prior art techniques.

FIG. 5 depicts the average change, between the initial and final points in 12 hours, of apical cornea thickness for six pairs of corneas for the prior art when compared with the exemplary embodiments of the disclosed subject matter.

FIG. 6 depicts the average change between the initial and final points in 12 hours, of effective refractive power for five pairs of loaded and not loaded corneas for the prior art when compared with the exemplary embodiments of the disclosed subject matter.

FIG. 7 depicts a representative time-history of effective refractive power variations over 12 hours for a pair of loaded and non-loaded corneas for the prior art when compared with the exemplary embodiments of the disclosed subject matter.

FIGS. 8A-8C depict representative refractive power maps from topographic measurements using exemplary embodiments of the disclosed subject matter.

FIGS. 8D-8F depict representative refractive power maps from topographic measurements using a control.

FIGS. 9A-9C depict representative OCT scans at apex cross section using exemplary embodiments of the disclosed subject matter.

FIGS. 9D-9F depict representative OCT scans at apex cross section using a control.

FIG. 10 depicts the average change between the initial and final points in 12 hours, of apical cornea thickness for five pairs of loaded and not loaded corneas for the prior art when compared with the exemplary embodiments of the disclosed subject matter.

FIG. 11A is a time plot illustrating the change in refractive power for a flattening procedure depicted in FIG. 11B.

FIG. 11B is a schematic drawing in which the cornea is deformed in a flattening procedure.

FIG. 12A is a time plot illustrating the change in refractive power for a steepening procedure depicted in FIG. 12B.

FIG. 12B is a schematic drawing in which the cornea is deformed with the coverslip and simultaneously irradiated with the femtosecond laser. The central region of the cornea remains intact, and the annulus shaped peripheral region is cross-linked.

FIG. 13 depicts the average change of effective refractive power (Diopter), between the initial and final measurement points for simultaneous cross-linking and mechanical loading (left) and for mechanical loading only (right) for six eye pairs using topography. (Error bars are standard deviations for six different corneas in each group.)

FIG. 14 depicts the average change of effective refractive power (Diopter), between the initial and final measurement points for simultaneous cross-linking and mechanical loading (left) and for mechanical loading only (right) for four eye pairs using OCT. (Error bars are standard deviations for four different corneas in each group.)

FIGS. 15A-15F are representative time histories of topography effective refractive power (Eff.RP) variations over 12 hours for a pair of corneas in which one of the corneas is subjected to the cross-linking and loading, and the other cornea is only subjected to the load.

FIGS. 16A-16E are representative time-histories of OCT diopter variations over 12 hours for a pair for corneas. Treated eye was subjected to simultaneous mechanical loading and CXL, and paired control was exposed to mechanical load only.

FIG. 17 is a representative time-history of effective refractive power variations over 12 hours for a pair for corneas. Treated eye was subjected to simultaneous mechanical loading and crosslinking (CxL), and paired control was exposed to mechanical load only. (Error bars are standard deviations for multiple topography data points taken at each time point for FIGS. 15A-15F, 16A-16E and 17.)

FIGS. 18A-18C are representative refractive power maps from topographic measurements. Cross-linked cornea is shown at FIG. 18A one hour, FIG. 18B right after crosslinking and deformation, and FIG. 18C at 12 hours after crosslinking and deformation.

FIGS. 18D-18F are representative refractive power maps from topographic measurements for control cornea at FIG. 18D one hour, FIG. 18E right after crosslinking and deformation, and FIG. 18F at 12 hours after crosslinking and deformation.

FIGS. 19A-19C are representative OCT scan at apex cross section. Cross-linked cornea is shown at FIG. 19A after one hour, FIG. 19B right after crosslinking and deformation, and FIG. 19C after at 12 hours after crosslinking and deformation.

FIGS. 19D-19F are representative OCT scan at apex cross section. Control cornea is shown at FIG. 19D after one hour, FIG. 19E right after crosslinking and deformation, and FIG. 19F after at 12 hours after crosslinking and deformation.

FIG. 20 depicts the average change, between the initial and final points in 12 hours, of apical cornea thickness for 6 pairs for cross-linked and not cross-linked corneas; all corneas are loaded during CXL. (Error bars are standard deviations for 6 different corneas in each group.)

FIG. 21 depicts the average change, between the initial and final points in 12 hours, of effective refractive power for 6 pairs of loaded and not loaded corneas during CXL. (Error bars are standard deviations for 6 different corneas in each group.)

FIG. 22 depicts a representative time-history of effective refractive power variations over 12 hours for a pair of loaded and not loaded corneas during CXL. (Error bars are standard deviations for multiple topography data points taken at each time point.)

FIG. 23A-23C are representative refractive power maps from topographic measurements. Cross-linked cornea is shown at FIG. 23A one hour, FIG. 23B right after crosslinking and deformation, and FIG. 23C at 12 hours after crosslinking and deformation.

FIGS. 23D-23F are representative refractive power maps from topographic measurements for control cornea at FIG. 23D one hour, FIG. 23E right after crosslinking and deformation, and FIG. 23F at 12 hours after crosslinking and deformation.

FIGS. 24A-24C are representative OCT scan at apex cross section. Cross-linked cornea is shown at FIG. 24A after one hour, FIG. 24B right after crosslinking and deformation, and FIG. 24C after at 12 hours after crosslinking and deformation.

FIGS. 24D-24F are representative OCT scan at apex cross section. Control cornea is shown at FIG. 24D after one hour, FIG. 24E right after crosslinking and deformation, and FIG. 24F after at 12 hours after crosslinking and deformation.

FIG. 25 depicts the average change, between the initial and final points in 12 hours, of apical cornea thickness for 5 pairs of loaded and not loaded corneas during CXL. (Error bars are standard deviations for 6 different corneas in each group.)

FIG. 26 is a schematic drawing of the techniques in accordance with a further embodiment of the disclosed subject matter.

FIG. 27 depicts Electron Paramagnetic Resonance (EPR) spectrum.

FIG. 28 depicts the oxidative modification of tyrosine.

FIG. 29 depicts the fluorescence spectrum of laser-treated and control samples of 5 mM tyrosine solution.

FIG. 30 depicts the thermal denaturation temperature of treated and control samples.

FIGS. 31-33 depict out of plane displacement maps showing displacement in the direction of the optical axis W (mm) for laser-treated corneas.

FIGS. 34-36 depict out of plane displacement maps showing displacement in the direction of the optical axis W (mm) for control corneas.

FIG. 37 depicts an out of plane displacement map showing displacement in the direction of the optical axis W (mm) for a control cornea.

FIG. 38 depicts an out of plane displacement map showing displacement in the direction of the optical axis W (mm) for 2-layer treated cornea. Laser treated region is circled

FIG. 39 depicts an out of plane displacement map showing displacement in the direction of the optical axis W (mm) for 5-layer treated cornea. Laser treated region is circled

FIG. 40 depicts hysteresis curve of apex (treated) and peripheral (control) regions of the cornea for control cornea.

FIG. 41 depicts hysteresis curve of apex (treated) and peripheral (control) regions of the cornea for 2-layers treated cornea.

FIG. 42 depicts hysteresis curve of apex (treated) and peripheral (control) regions of the cornea for 5-layers treated cornea.

FIG. 43 depicts a two-photon fluorescence (TPF) image of an untreated control rabbit cornea.

FIG. 44 depicts a two-photon fluorescence (TPF) image of an anterior laser treated rabbit cornea treated ex vivo.

FIG. 45 depicts a two-photon fluorescence (TPF) image of a posterior laser treated rabbit cornea treated ex vivo.

FIG. 46 depicts the average pixel value for the corneas shown in FIGS. 43-45.

FIG. 47 depicts effective refractive power (ERP) of the eye and comparing it to a control eye over several weeks.

FIG. 48A depicts a histological section of a hematoxylin-eosin (H&E) stained rabbit cornea at 48 hours after treatment. (scale bar 100 μm.)

FIG. 48B depicts a histological section of a hematoxylin-eosin (H&E) stained rabbit cornea at 7 days after treatment. (scale bar 100 μm.)

FIG. 48C depicts a histological section of a hematoxylin-eosin (H&E) stained rabbit cornea at 3 months after treatment. (scale bar 100 μm.)

FIG. 48D depicts a histological section of a hematoxylin-eosin (H&E) stained rabbit cornea at 48 hours after treatment for untreated control (scale bar 100 μm).

FIG. 48E depicts a histological section of a hematoxylin-eosin (H&E) stained rabbit cornea at 7 days after treatment for untreated control (scale bar 100 μm).

FIG. 48F depicts a histological section of a hematoxylin-eosin (H&E) stained rabbit cornea at 3 months after treatment for untreated control (scale bar 100 μm).

FIG. 49A depicts in vivo confocal microscopy images of laser-treated epithelial tissue of rabbit eyes obtained 48 hours after treatment (scale bar 50 μm).

FIG. 49B depicts in vivo confocal microscopy images of laser-treated keratocyte tissue of rabbit eyes obtained 48 hours after treatment (scale bar 50 μm).

FIG. 49C depicts in vivo confocal microscopy images of laser-treated endothelium tissue of rabbit eyes obtained 48 hours after treatment (scale bar 50 μm).

FIG. 49D depicts in vivo confocal microscopy images of control epithelial tissue of rabbit eyes obtained 48 hours after treatment (scale bar 50 μm).

FIG. 49E depicts in vivo confocal microscopy images of control keratocyte tissue of rabbit eyes obtained 48 hours after treatment (scale bar 50 μm).

FIG. 49F depicts in vivo confocal microscopy images of control endothelium tissue of rabbit eyes obtained 48 hours after treatment (scale bar 50 μm).

FIGS. 50-51 depict an ex vivo experimental setup, in which the eye is mounted onto a custom built holder, applanated with a microscope cover slip, and attached to IV system in accordance with exemplary embodiments of the disclosed subject matter.

FIG. 52 depicts a schematic of an exemplary method of the disclosed subject matter.

### DETAILED DESCRIPTION

Embodiments of the invention provide methods and systems for treatment of tissue, e.g., within a cornea. Embodiments of the invention can be applied to a variety of tissue including cartilage, articular cartilage, and the like. Reference will now be made in detail to select embodiments of the disclosed subject matter, examples of which are illustrated in the accompanying drawings. The method and corresponding steps of the disclosed subject matter will be described in conjunction with the detailed description.

Unless defined otherwise, all technical and scientific terms used herein have the same meaning as commonly understood by one of ordinary skill in the art to which this disclosed subject matter belongs. Although any methods and materials similar or equivalent to those described herein can also be used in the practice or testing of the present disclosed subject matter, this disclosure may specifically mention certain exemplary methods and materials.

As used herein and in the appended claims, the singular forms “a,” “an,” and “the” include plural referents unless the context clearly dictates otherwise.

Collagen crosslinking in the cornea has the capability of enhancing its mechanical properties and thereby providing an alternative treatment for eye diseases such as keratoconus. In addition, selective crosslinking together with mechanical deformation allows for corneal reshaping for vision correction. If a mechanical load is applied prior to and concurrently with the crosslinking procedure, the corneal curvature can be reshaped, and thus correct refractive errors. It is believed that pre-loading allows for corneal reshaping, while the crosslinking permanently ‘locks’ the deformation. The procedure is independent of the crosslinking methodology, and can be achieved with laser-based crosslinking, chemical crosslinking, UV/riboflavin crosslinking, etc.

In one aspect, a technique for treatment of the cornea includes exposing the cornea to a crosslinking medium, applying a mechanical loading to the cornea, wherein the mechanical loading is selected proportional to the dimensions of the eye being treated. In some embodiments, the mechanical loading is a 15% deformation (measured by a micrometer) of the original eye ball height. In some embodiments, the mechanical loading was achieved using a 1.6 mm diameter cylinder stick. In some embodiments, the crosslinking medium includes UV-A light and riboflavin.

By controlling (a) the strain (deformation), e.g., as a percentage of the size of the eye, and (b) the size of the deformed region (currently 1.6 mm in the apex region of the cornea), it is possible to adjust vision correction outcomes.

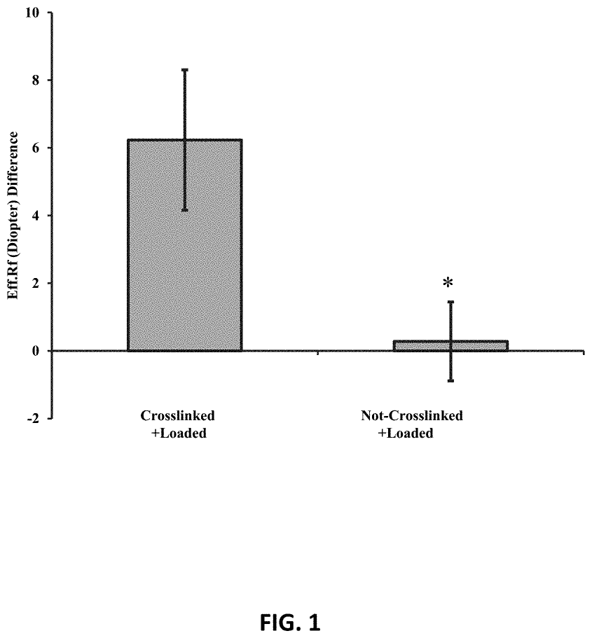

Experiments shown in FIGS. 1-5 confirm that a mechanical load, applied together with crosslinking, is a necessary component to achieve diopter change. In the experiments, a mechanically loaded and crosslinked eye has been compared against a control that was subjected to mechanical loading only. Control eyes had some deformation initially, but after about 12 hours, the corneal curvature (diopter) recovered to pre-deformation state. On the other hand, corneas subjected to mechanical load AND crosslinking remained deformed.

EXPERIMENT A: The first experiment, shown in FIGS. 1-5, demonstrates that mechanical deformation alone does not provide long-lasting diopter change.

Rabbit eyes were harvested from freshly sacrificed animals. Paired eyes were dissected form the eye sockets with the optical nerve head intact. The epitheliums were removed using an electric rotatory brush. The height of the eye ball from the apex of the cornea to the optical nerve head was measured using a caliper. The eyes were then transferred to a custom made holder and connected to IV system to maintain a pressure of 20 mm Hao at all times. Effective refractive power was measured using Eyesys Vista hand held topography system. For pink rabbit eyes, Trypan Blue was used for easier image recognition for Eyesys Vista. Cross sectional scanning from optical coherence tomography was taken using Lumedica OQ Labscope version 1.5. All data point collection included both topography and OCT.

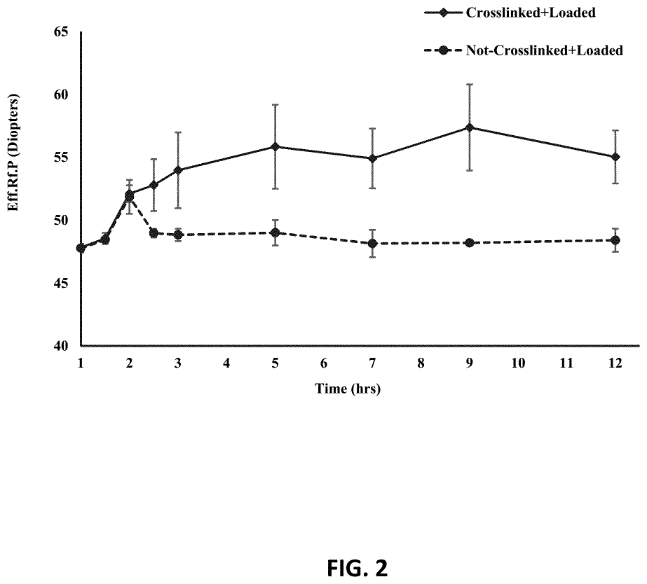

Eyes were allowed to rest in the holder for one hour to allow pressure stabilization. After that one hour, the first data point was taken. A typical UV-A light/riboflavin crosslinking (hereinafter “CXL”) was then performed on the treated eye. The procedure began by applying 0.1% riboflavin in 20% Dextran 500 (Sigma) drops at 5 minute intervals for 20 minutes. The second data point was taken (FIG. 2), before the entire cornea was irradiated with UV-A light (365 nm) for 30 minutes with an irradiance of approximately 3 mW/cm² (Crystal BioGrow UV-Lamp, Model: BG-32a), while 0.1% riboflavin was continued to be applied onto the cornea every 5 minutes.

The distance between the corneal apex and UV-A light window was kept between 35 to 50 mm. During the 30 minutes exposure time to UV-A light, a central loading was applied such that a 15% deformation (measured by a micrometer) of the original eye ball height was achieved using a 1.6 mm diameter cylinder stick. Immediately after CXL treatment, the third data point was taken. The fourth and the fifth data points were taken 30 minutes apart following the third point, and after that each data point was taken approximately 2-3 hours apart until 12 hours are reached. For the paired control eye, all protocol was the same as one for the treated eye, but without UV-A light application.

FIG. 1 illustrates the averaged difference, before treatment and after 12 hours, of effective refractive power (Diopter) for five pairs of corneas. The measurements were taken at regular intervals over span of 12 hrs. All corneas were subjected to mechanical loading. Treated corneas were mechanically loaded and crosslinked (CxL), whereas control corneas were only subjected to mechanical load. Mechanical load and CxL was simultaneous. Error bars are standard deviations for six different corneas in each group. A paired one tailed t-test shows the difference between the crosslinked and not-crosslinked group is statistically significant (P<0.05).

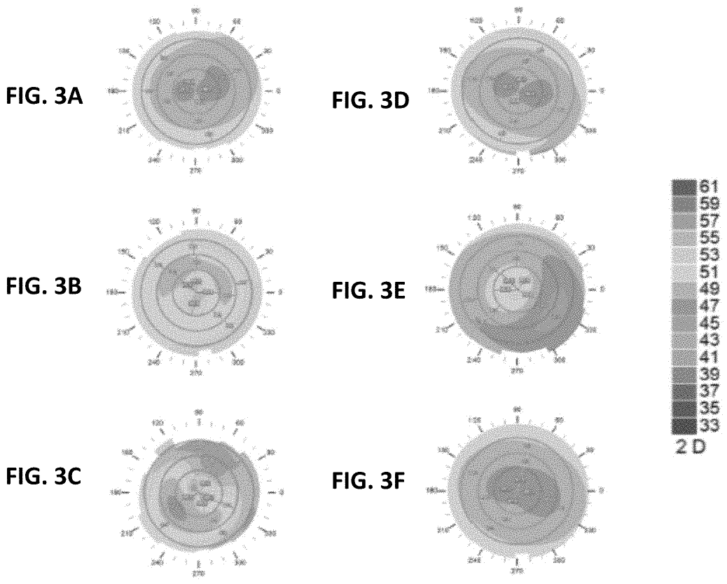

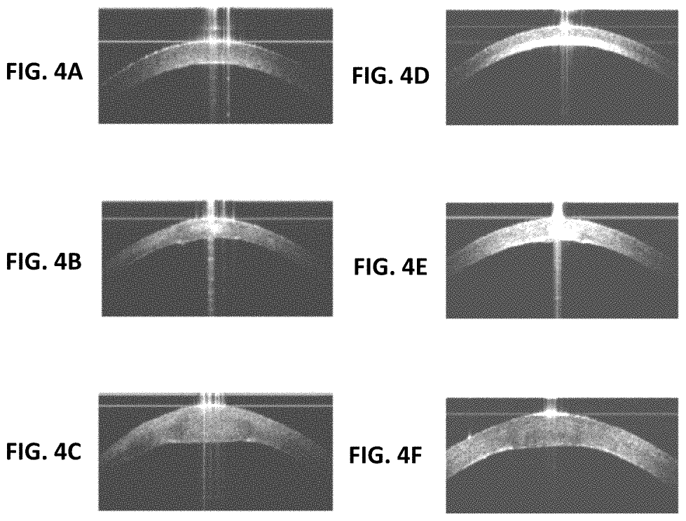

One pair of the rabbit corneas is chosen as the representative to show the time-history of the effective refractive power (FIG. 2), the refractive map from topography (FIG. 3), and the apex cross sectional scanning from optical coherence tomography (OCT) (FIG. 4).

FIG. 2 illustrates representative time-history of effective refractive power variations over 12 hours for a pair for corneas. Treated eye was subjected to mechanical loading and crosslinking (CxL), and paired control was exposed to mechanical load only. Mechanical load and CxL was simultaneous. Error bars are standard deviations for multiple topography data points taken at each time point.

FIGS. 3A-3F illustrate representative refractive power maps from topographic measurements. Crosslinked cornea is shown at various times (FIG. 3A) one hour, (FIG. 3B) right after crosslinking and deformation, and (FIG. 3C) 12 hours. Corresponding control cornea (no CxL applied) is shown at (FIG. 3D) one hour, (FIG. 3E) right after deformation, and (FIG. 3F) 12 hours. FIGS. 4A-4F illustrate representative OCT scans at apex cross section. Crosslinked cornea is shown at (FIG. 4A) one hour, (FIG. 4B) right after crosslinking and deformation, and (FIG. 4C) 12 hours. Not-crosslinked control cornea is shown at (FIG. 4D) one hour, (4E) right after deformation, and (FIG. 4F) 12 hours.

After the application of a central loading, both UV-A light riboflavin-treated cornea and its paired control have a sudden increase in refractive power due to an apical deformation, which can be observed from both the center of topography (FIG. 3B and FIG. 3E) and OCT (FIG. 4B and FIG. 4E). After treatment until the 12-hour time point, the deformation of the crosslinked eye (FIG. 3C and FIG. 4C) becomes more pronounced than its paired non-crosslinked control (FIG. 3G and FIG. 4G). The UV-A light riboflavin treated cornea maintains its increase in diopters, whereas the control cornea's refractive power drops back to the starting point.

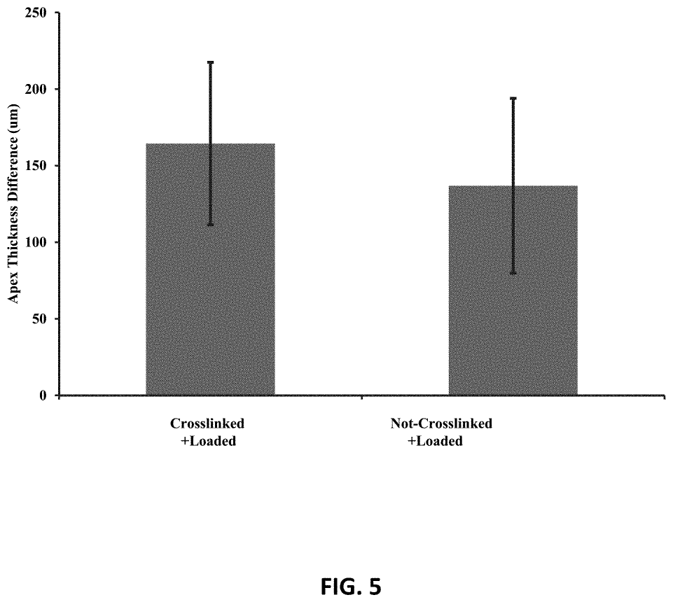

FIG. 5 illustrates the average change, between the initial and final points in 12 hours, of apical cornea thickness for 6 pairs for crosslinked and not-crosslinked corneas; all corneas are loaded during CXL. Error bars are standard deviations for six different corneas in each group.

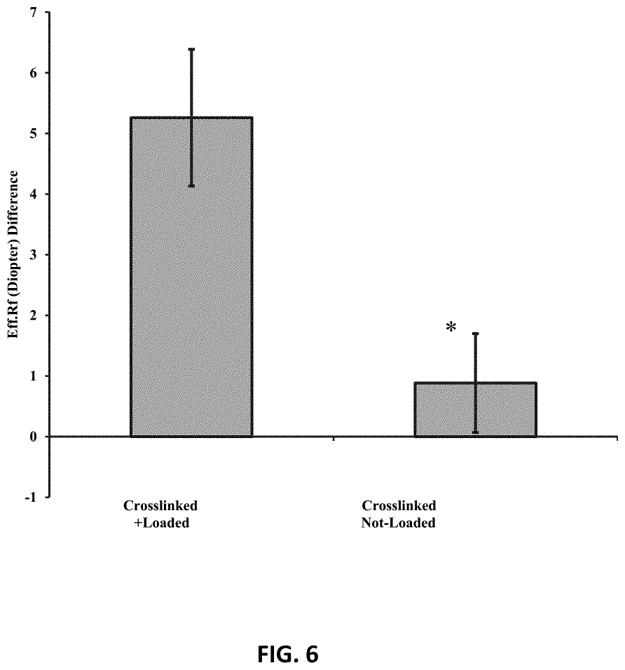

EXPERIMENT B. The second experiment, shown in FIGS. 6-10, demonstrates that crosslinking alone has little effect on the diopter change.

Paired eyes were crosslinked, however one was subjected to mechanical load AND crosslinking, whereas the second eye was only cross linked, as in conventional practice. The eye that was mechanically deformed AND cross linked has significant diopter change. In contrast, the paired control (crosslinking only) has not had much of diopter change.

FIG. 6 illustrates the average change between the initial and final points in 12 hours, of effective refractive power for 5 pairs of loaded and not loaded corneas during CXL. Error bars are standard deviations for 5 different corneas in each group.

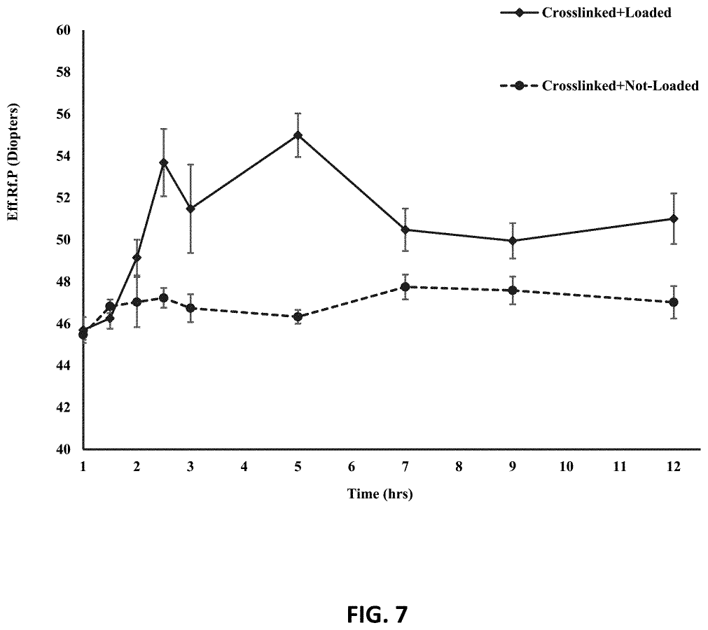

FIG. 7 illustrates a representative time-history of effective refractive power variations over 12 hours for a pair of loaded and non-loaded corneas during CxL. Error bars are standard deviations for multiple topography data points taken at each time point.

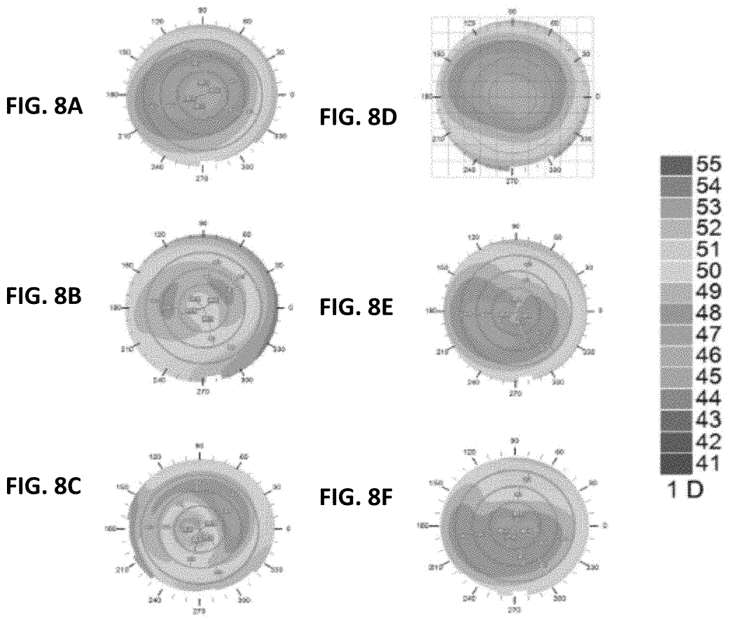

FIGS. 8A-8F illustrate representative refractive power maps from topography. Loaded cornea is shown at (FIG. 8A) one hour, (FIG. 8b) right after crosslinking and deformation, and (FIG. 8c) 12 hours. Not-loaded control cornea is shown at (FIG. 8d) one hour, (FIG. 8e) right after deformation, and (FIG. 8f) 12 hours.

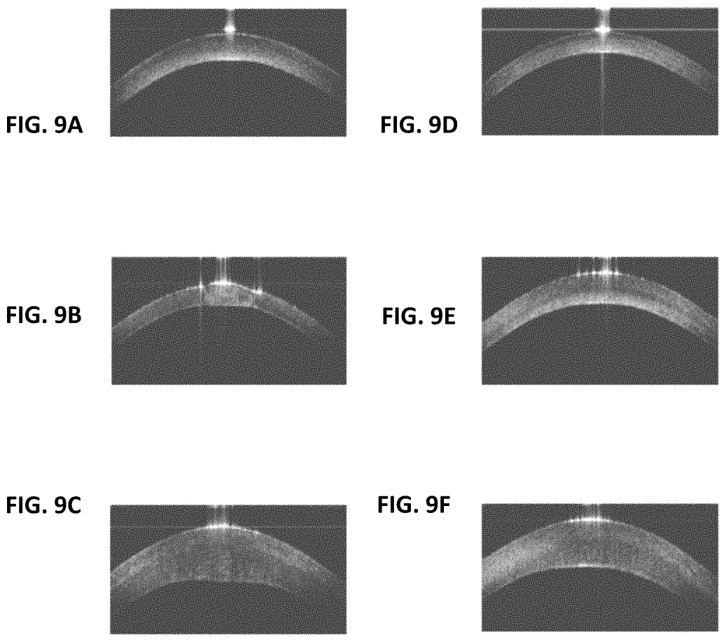

FIGS. 9A-9F illustrate representative OCT scans at the apex cross-section. Loaded cornea is shown at (FIG. 9A) one hour, (FIG. 9B) right after crosslinking and deformation, and (FIG. 9C) 12 hours. Non-loaded control cornea is shown at (FIG. 9D) one hour, (FIG. 9E) right after deformation, and (FIG. 9F) 12 hours.

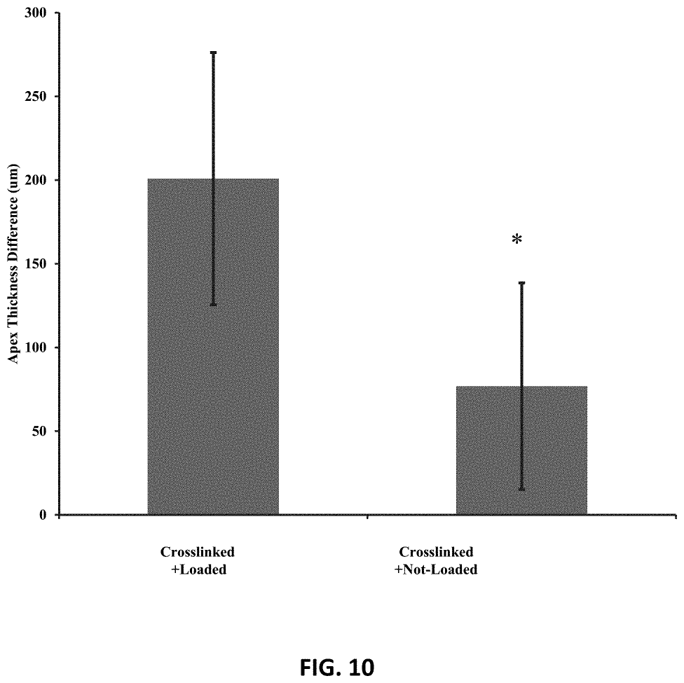

FIG. 10 illustrates the average change between the initial and final points in 12 hours, of apical cornea thickness for 5 pairs of loaded and not loaded corneas during CXL. Error bars are standard deviations for 5 different corneas in each group. The eye that was mechanically deformed AND cross linked maintains a significant diopter change. In contrast, the paired control (crosslinking only) did not maintain as much of diopter change.

Experimental Research: Two main observations have been noted from the data: (1) most of the control eyes exhibited no flattening (change in diopter due mechanical deformation), and (2) one experiment (two porcine eyes) in which deformed cornea was cross-linked with riboflavin/UVA light had significant and dramatic change in diopter. Note: riboflavin/UVA light standard crosslinking procedure: epithelium is removed, eye soaked with riboflavin solution and exposed to 370 nm wavelength light.

In some experiments, the change in effective refractive power is achieved by mechanically deforming the cornea first, and then ‘locking’ the deformation with crosslinking. Corneal flattening (myopia treatment) is achieved by pressing the cornea with a cover slip, and then irradiating its central region with femtosecond laser while cornea was still deformed.

In other experiments, steepening techniques (hyperopia treatment) have been used, in which the same cover slip-induced mechanical loading was applied as in the corneal flattening experiments, but the laser irradiation pattern was annulus shaped, such that apical region of cornea remained intact while the peripheral region was treated with the laser.

Two challenges have been identified with corneal reshaping experiments: (a) the corneas were not sufficiently deformed to allow for the treatment based diopter change, and (b) the laser was not crosslinking stromal tissue. The disclosed technique decouples these two problems. Thus, in the disclosed subject matter, crosslinking combined with appropriate mechanical load results in corneal reshaping (diopter change), regardless the source of crosslinking.

Crosslinking combined with mechanical load results in corneal reshaping: Some studies attempt to discuss crosslinking pathways, and most research papers identify singlet oxygen as culprit in CxL. However, Sel et al., “UVA irradiation of riboflavin generates hydroxyl radicals”, Redox Report 19(2), 72-79 2014, has shown that application of riboflavin/UVA light in 0.9% NaCl produces hydroxyl radicals. Electron spin resonance spectroscopy (EPR) with DMPO as spin-trapper was utilized their experiments.

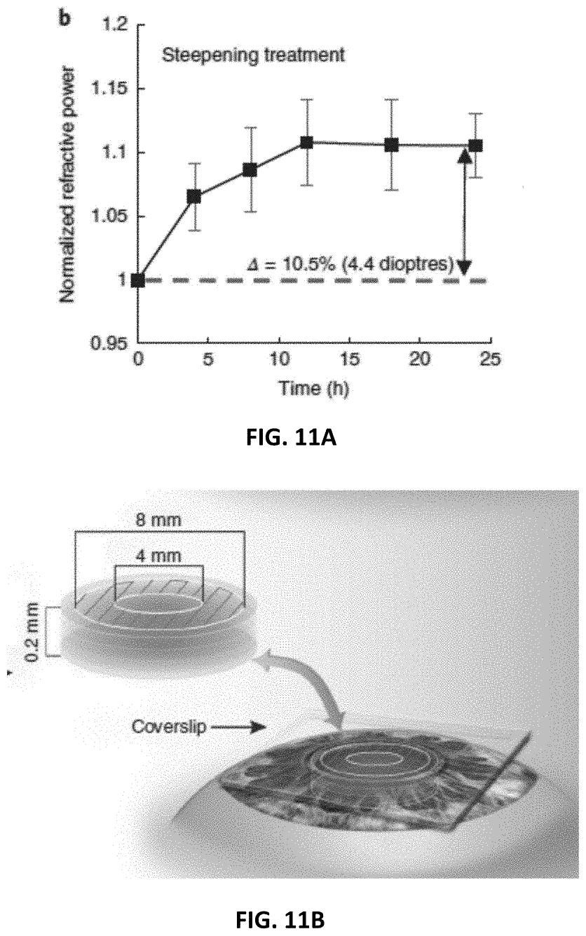

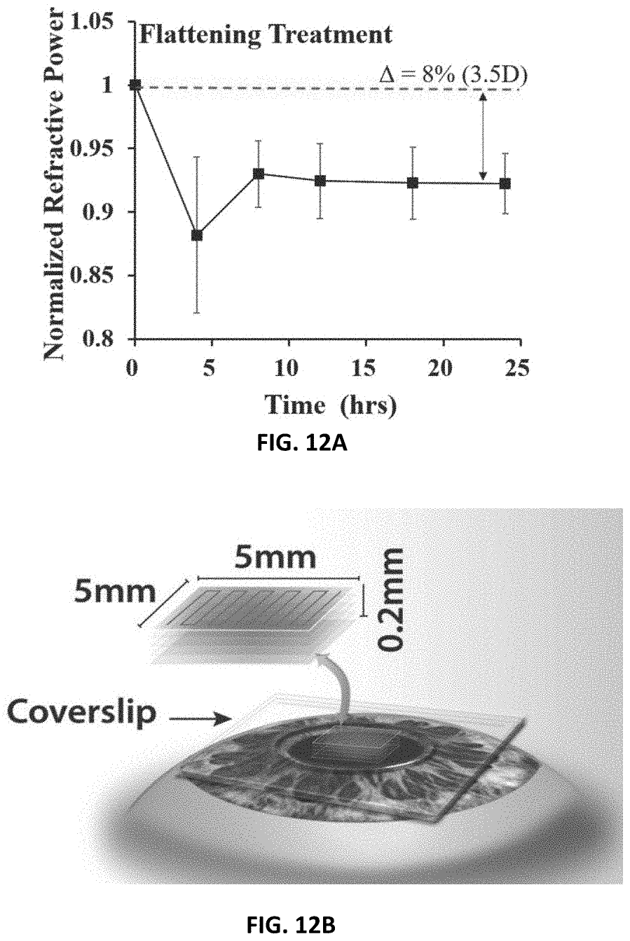

In accordance with exemplary embodiments, a laser source is replaced with a reliable crosslinking agent, and all other criteria for corneal reshaping are met, a diopter change in treated eyes is achieved. As discussed further below, the cornea treatments include a flattening experiment, a steepening procedure and a control. In the flattening experiment (FIGS. 11A-11B), e.g., for treatment of myopia, the corner was flattened with a cover slip and cross-linked. In the steepening procedure (FIGS. 12A-12B), e.g., for treatment of hyperopia, the cornea is mechanically loaded, during crosslinking, and the central region of the cornea is covered with the pin, and thus not cross-linked (no exposure to UVA light), while the annulus shaped peripheral region is cross-linked.

Methods: The protocol for corneal steepening using riboflavin/UVA as a source of CxLs is set forth herein. An exemplary objective is to apply mechanical load and CxL in peripheral region while leaving central cornea chemically intact.

Use of cover slip was impractical as it limits oxygen availability. Instead, a Æ1.6 mm disc has been used to apply loading, and mask the central region from UVA light. A total of 12 pairs of fresh rabbit eyes were enucleated and their epitheliums removed. Eyes were placed in a custom built holder, and the intraocular pressure was kept at 20 mm H2O with IV system. Six pairs were assigned to Group 1, and remaining six to Group 2.

Group 1: Each treated eye in Group 1 was subjected to typical Riboflavin/UVA CxL procedure, together with a 2E1.6 mm central loading applied such that 15% deformation, measured as change in the distance between the corneal apex and the optical nerve head, was achieved. Paired controls in Group 1 were subjected to the identical mechanical load and riboflavin solution, but were not exposed to UV-A light.

Group 2: In Group 2 treated eye of each pair was subjected to the same CxL and mechanical load procedure as treated eyes in Group 1. However, paired controls in Group 2 were cross-linked, following a typical Riboflavin/UVA CxL procedure, but received no mechanical loading.

Corneal topography and optical coherence tomography (OCT) were taken over a 12-hour period to collect effective refractive power (Eff.RP) and apical corneal thickness. Paired t-tests were used for statistical analysis. Two operators independently collected data with topographer at every measurement point (usually three measurements each), and there were always at least two people at the lab during the data collection. Saline solution was used during topography acquisition instead of Systane drops to ensure that coating of the eye with a viscous fluid does not skew results. Use of saline expedited corneal swelling because of which data was collected over 12 hours period instead of 24 hours. However, 12 hours appears to be sufficient time to capture trends.

Optical coherence tomography (OCT) volumetric scans were obtained simultaneously with topography time points over the span of 12 hours. The scans were used to calculate eye diopters using Labview files. Diopters were be calculated from curve fitting of the anterior corneal surface. However, due to the apical morphological changes introduced by the mechanical load, the OCT cross-sectional scans of anterior corneal surface are not smooth. Thus, the estimated fitting curve can sometimes be overly prolate (overestimating) or oblate (underestimating), leading to a difference in acquired diopters from topography measurements.

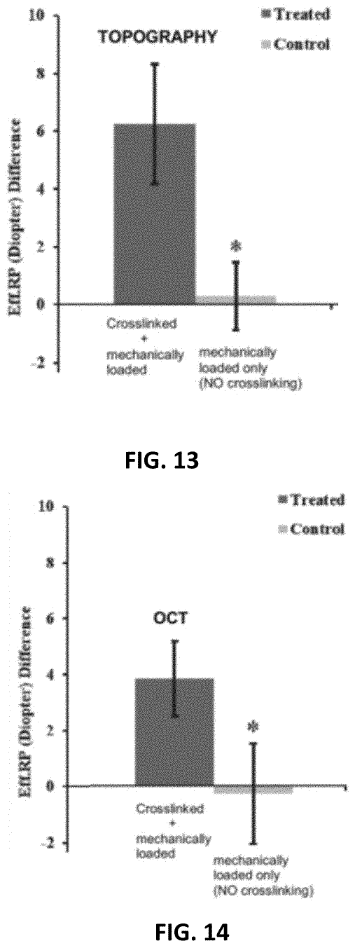

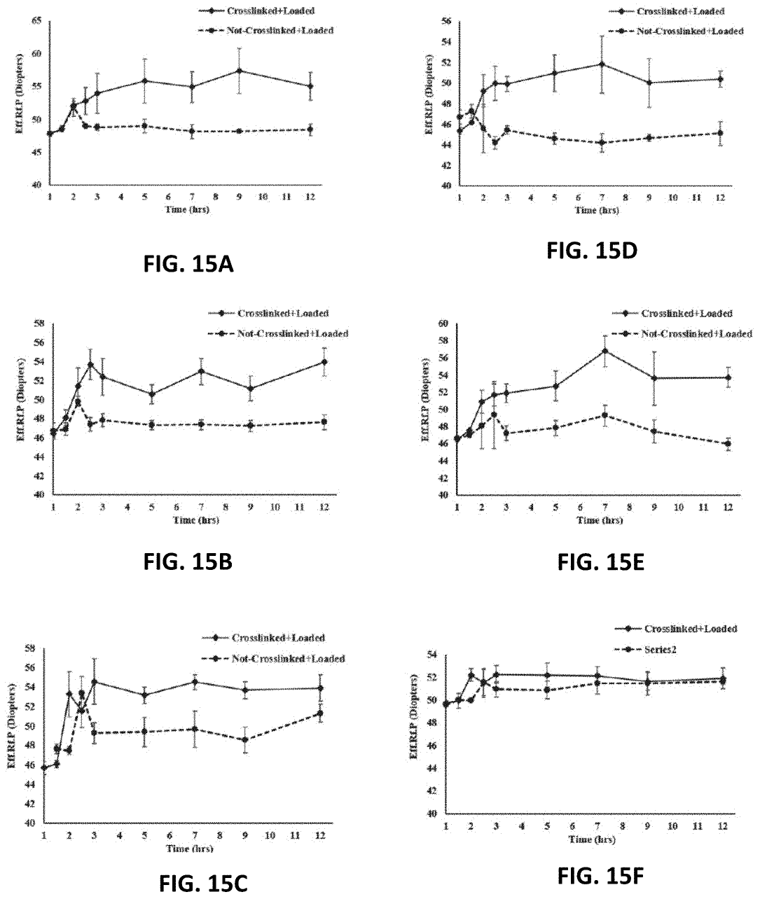

Results Group 1: FIGS. 13-14 summarize outcomes in Group 1. Topographic measurements show that cross-linked and loaded corneas exhibit average diopter change of 6 Diopter (D), whereas there is no significant diopter change in paired controls that were subject to mechanical load only. Significant diopter change between treated eyes and paired controls was observed in 5 out of 6 pairs after 12 hrs. Both eyes in the eye pair in which there was no significant difference in diopter between the treated and control eye had somewhat higher initial diopter than other pairs in Group 1.

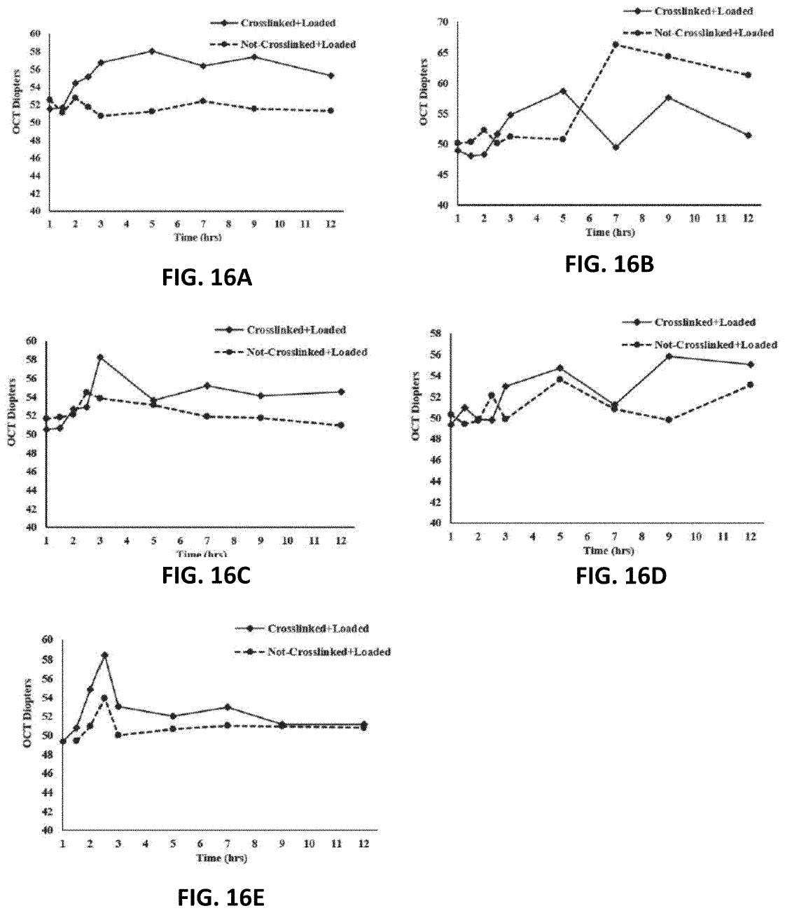

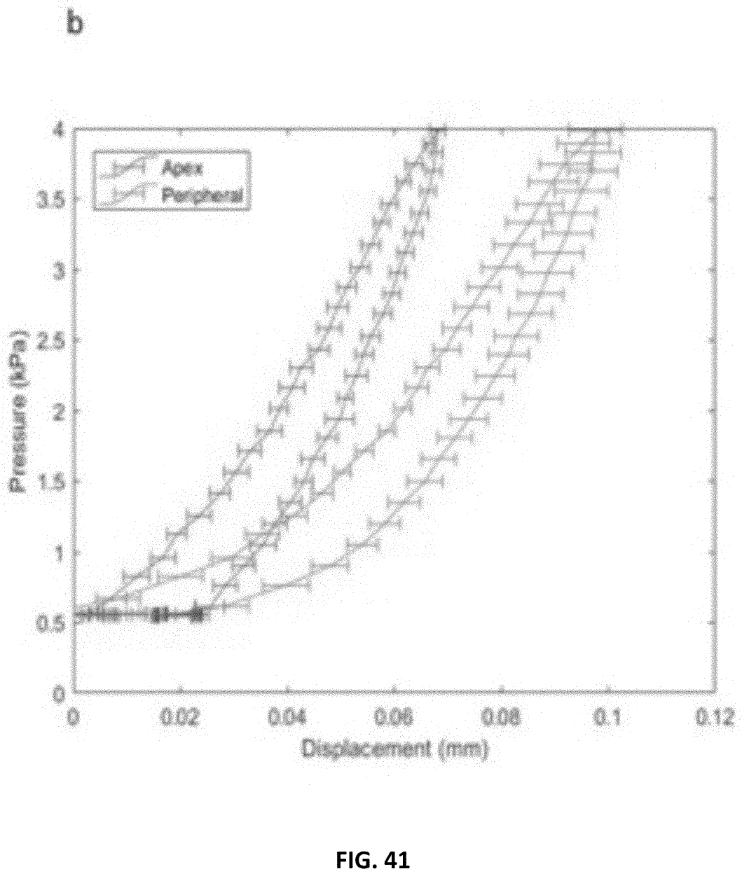

Time-history of diopter change over 12 hours for all 6 pairs can be found in FIGS. 41A-41F. OCT data includes results from 4 out of 6 pairs. One data set has not been processed yet, however raw data (along with all other raw data for these experiments) is stored on the lab computer. This result will be added to the summary as soon as we regain access to the lab. One pair is excluded from the summary as issues were observed during data acquisition and processing. However, time-history of the diopter change of this particular pair can be found in FIG. 16B, together with diopter change time histories of all other pairs.

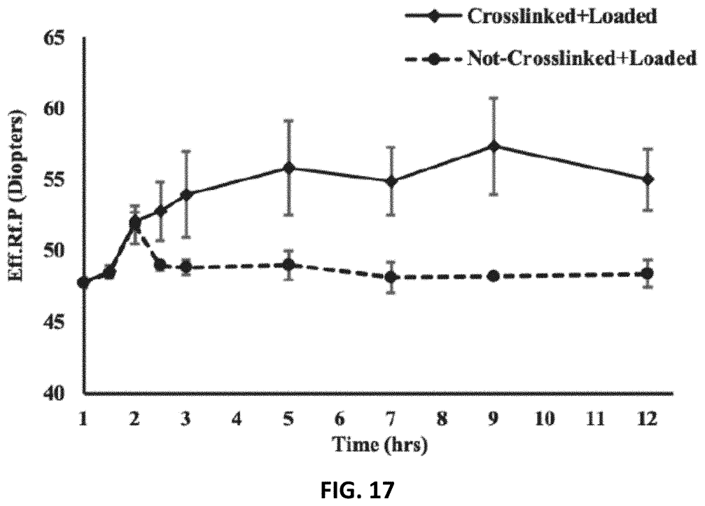

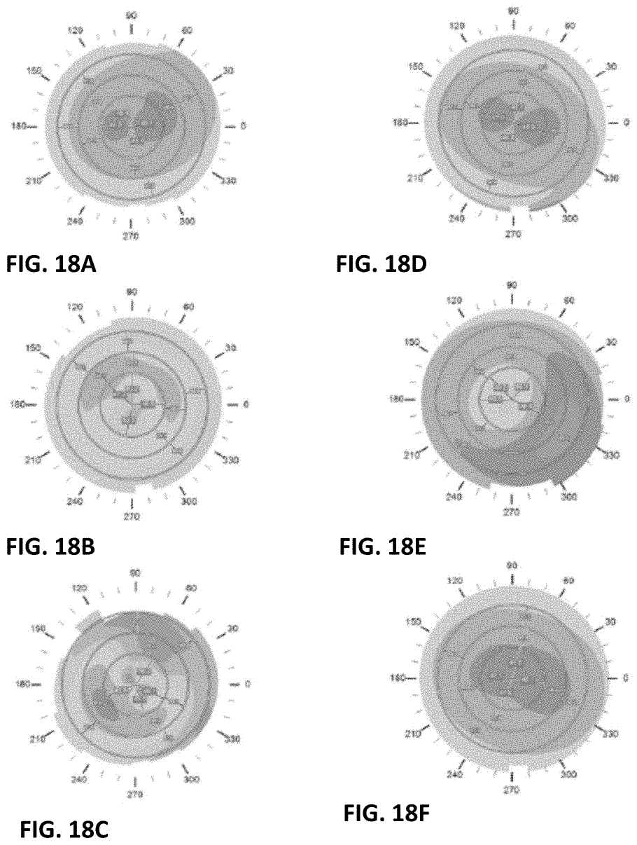

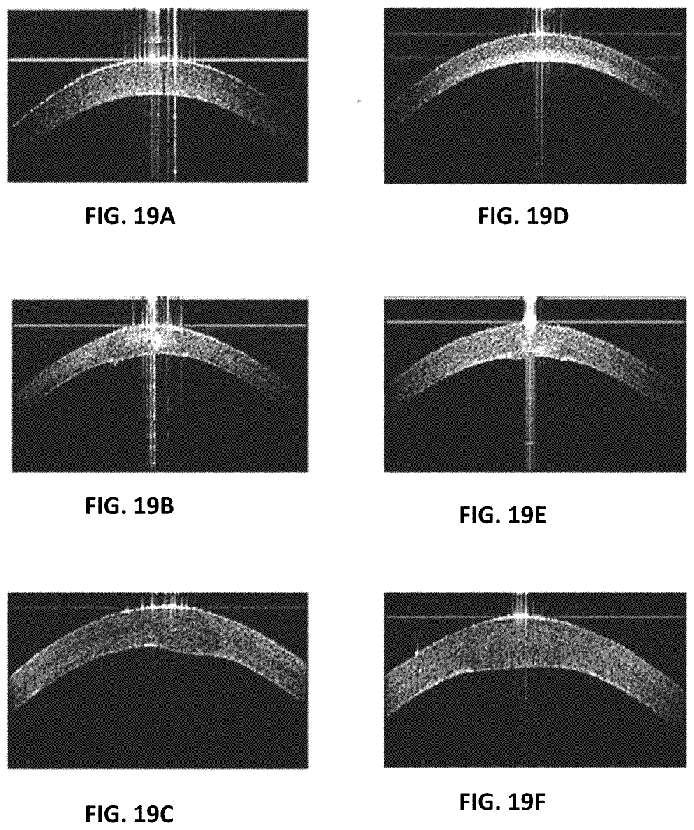

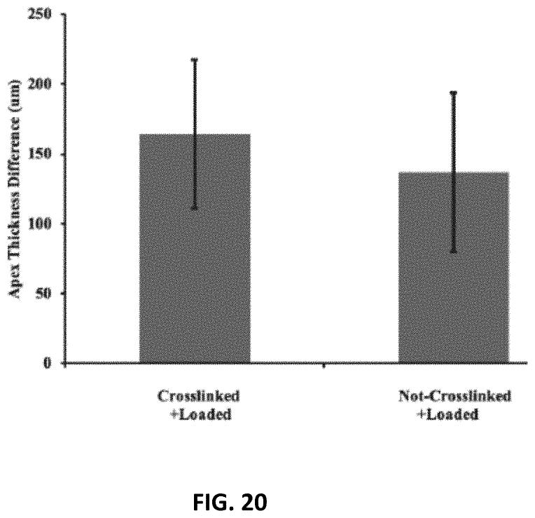

A representative time-history of the diopter change for Group 1 is shown in FIG. 17. It is interesting to note that the evolution of the diopter of treated corneas is similar to that seen in the femtosecond laser irradiated eyes (FIGS. 12-13). Paired controls have temporary Increase in Eff.RP that dissipates after about three hours. FIGS. 18A-18F depict representative refractive power maps from topographic measurements. FIGS. 19A-19F show representative OCT scans at apex cross section. FIG. 20 summarizes changes in corneal thickness of treated eyes and paired controls after 12 hours period.

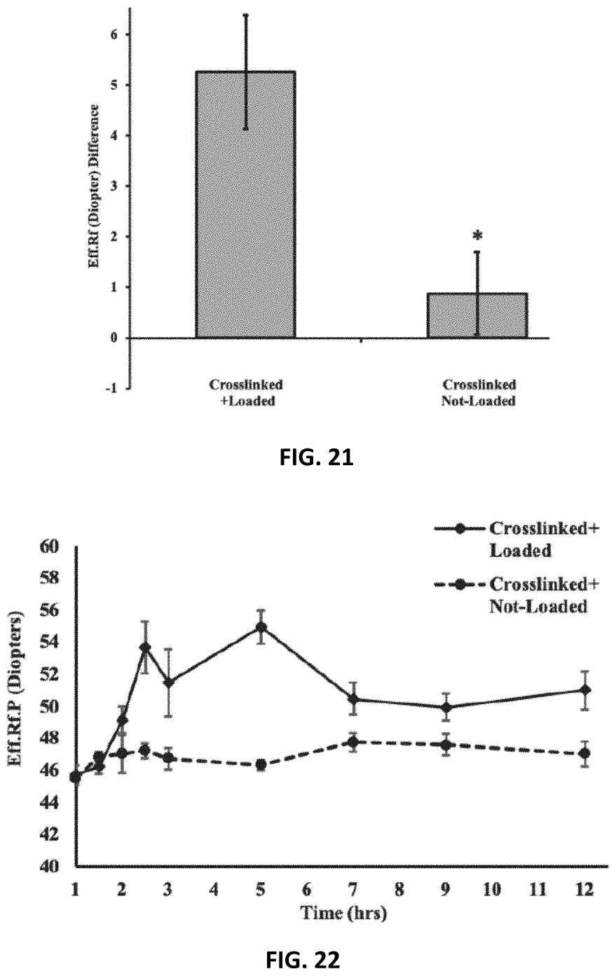

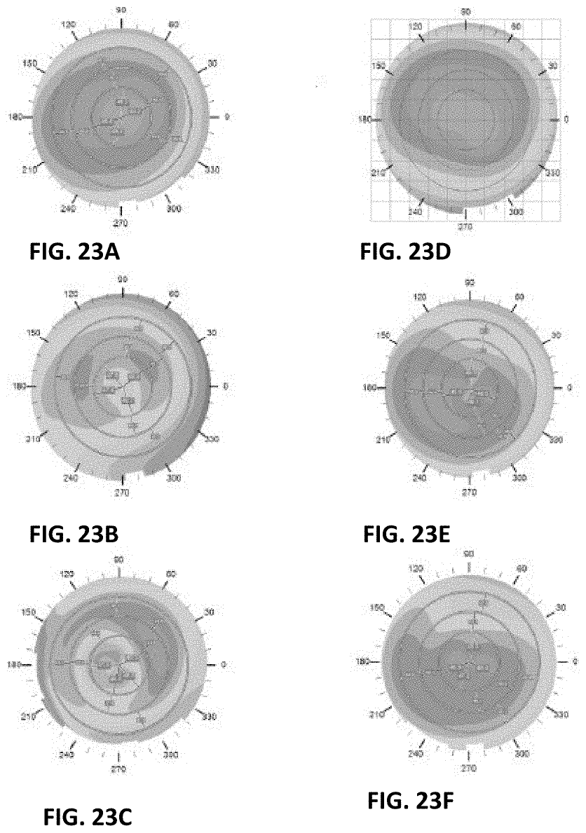

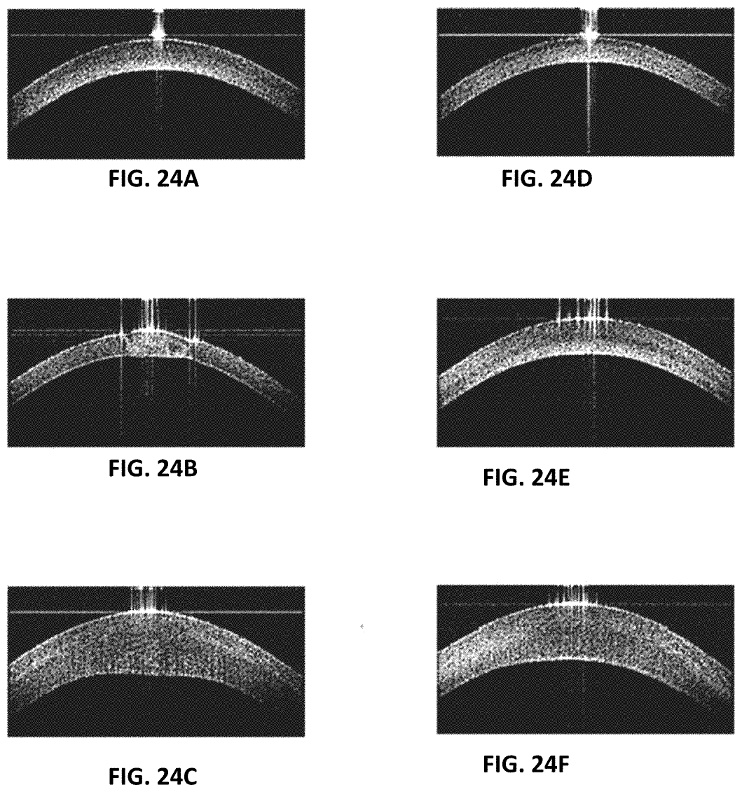

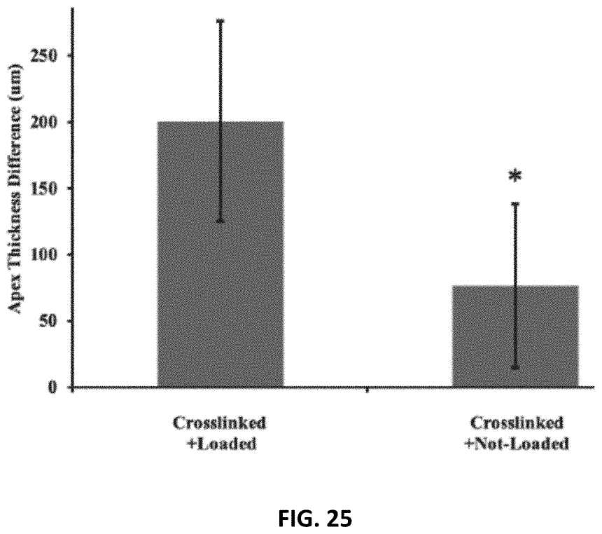

Results—Group 2: FIG. 21 summarizes outcomes in Group 2. The results from topography are shown here. Cross-linked and loaded corneas exhibit similar average diopter change as in Group 1, which is understandable as both are subject to the same conditions. Paired controls have some diopter change (˜1D), which is consistent with literature, and claims made by Avedro Inc. Time-history, representative topographic maps, OCT scans, and corneal thickness are shown in FIGS. 22, 23A-23F, and 24A-24F, respectively.

Conclusion: Mechanical loading is an important and complex component of the procedure that needs more attention. Initially, in the experiments that precede ones shown in this document, we have subjected corneas to a fixed displacement. In those early experiments the results were inconsistent, some eyes showed significant diopter change, while others did not deform much. Then we have realized that the inconstancy is related to eye size. After switching from fixed displacement, to deformation as a function of the size of the eye, results were much more consistent.

In another embodiment, a novel, laser-based treatment modality is used to retard and reverse KCN progression without use of photosensitizers, and with minimal or no adverse effects. The techniques described herein rely on the novel use of the femtosecond oscillator, a low power ultrafast laser, for selective and localized modification of biomechanical properties of corneal stroma, such that ionization of target molecules within cornea is achieved while avoiding damaging optical breakdown of the tissue.

A low-density plasma (LDP) is used to generate an ionization field within biological media without any damaging thermoacoustic and shock waves. When applied to collagen-rich tissues, LDP produces reactive oxygen species (ROS) by breaking down water. The resulting radicals react with surrounding proteins to form crosslinks. High precision of the procedure allows for spatially-resolved modification of mechanical properties. Fine tuning of mechanical properties within the cornea allows for personalized treatment, as well as adjustment of overall corneal curvature.

In treating KCN, CxL significantly increases corneal rigidity. However, both in vitro and in vivo cytotoxicity studies have shown that the procedure leads to an immediate loss of stromal keratocytes throughout the entire volume of the affected stroma. It can take up to six months to repopulate the corneal stroma with activated keratocytes, but the treatment is considered safe for corneas thicker than 400 μm. The main safety concern is the potential for endothelial damage. The endothelium of the healthy cornea plays a key role in maintaining corneal hydration and transparency via active sodium-potassium adenosine triphosphatase (ATPase) and bicarbonate-dependent magnesium ATPase ion pumps. The corneal endothelial cells cannot replicate, and compensation can be achieved only by sliding. If cell density decreases to levels below the critical limit, endothelial barrier may become dysfunctional, resulting in vision loss. Additional side effects include pain, and temporary loss of vision for few days after the treatment. Stromal haze is another side effect that develops weeks after the procedure and lasts to about 1-year post-treatment. Finally, CxL procedure is also associated with damage of corneal nerves, mostly due to epithelium removal.

In exemplary embodiments, the techniques described herein utilize an infrared ultrafast laser, in absence of photosensitizers. This eliminates cytotoxicity, and thus is much safer. Furthermore, extremely low laser pulse energy (˜1.2 nJ) is used, which does not inflict thermal damage.

Efforts to improve standard CxL include (a) speeding up the procedure, and (b) modifying the protocol to eliminate need for epithelium debridement. Reducing of the procedure time can be achieved by increasing the radiation light intensity from 3 mW/cm², to 10 mW/cm², which decreases the procedure duration from 30 to 9 minutes. For example, transepithelial CxL has been introduced to circumvent the epithelium removal while addressing poor riboflavin penetration through intact cornea. Much smaller increase in corneal stiffening occurs in transepithelial CxL when compared against standard clinical protocols.

The use of pulsed UVA light irradiation, rather than continuous beam, has been used and presumably allows for increased oxygen diffusion into the stroma, and thus enables Type II crosslinking mechanism to dominate the process. Although deeper demarcation zone is seen, apoptosis rates are higher and optimal pulsing protocol unclear. In another example, laser in situ Keratomileusis (LASIK) Xtra, surgery, when combined with CxL, addresses effects of surgical weakening of the cornea, and post-LASIK regression. However, concerns over combining CxL with LASIK include CxL induced flattening which diminishes the accuracy of the refractive correction and introduction of stromal haze. In yet another example, CxL is performed in absence of UVA light using Nonlinear (NLO) and chemical crosslinking. By adopting polymer microfabrication approach, in NLO corneal CxL, riboflavin has been activated with infrared femtosecond laser. Advantages of this approach is reduced toxicity, faster treatment time and spatially resolved CxL. However, the riboflavin penetration is still an issue. Use of chemical agents for scleral crosslinking has aimed to slow myopia progression by injecting a stabilizing agent via the sub-Tenon's space to CxL posterior sclera. Such an approach also eliminates the need for UVA light irradiation.

The techniques described herein incorporate a laser treatment in absence of photosensitizers on intact eyes to retard progression of KCN, while simultaneously improving patient's visual acuity. The techniques described herein exploit a radically different mode of laser-tissue interaction, which avoids both thermal ablation and optical breakdown, to demonstrate a novel approach to enhancement of mechanical properties of KCN affected eyes.

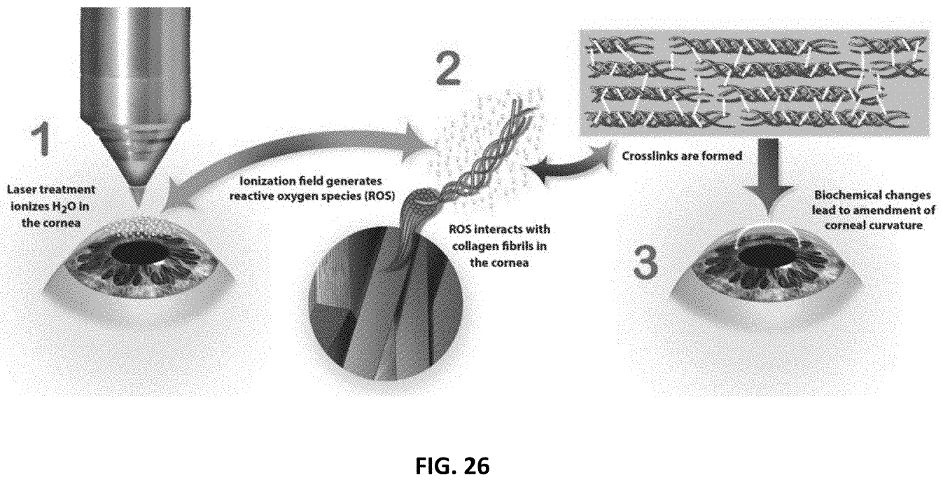

FIG. 26 illustrates the treatment process in accordance with an exemplary embodiment. In an early stage of the process, a femtosecond laser irradiates corneal stroma creating an ionization field.

In a later stage of the process, ionization generates reactive oxygen species (ROS).

In a further stage of the process, the ROS interact with collagen fibrils in the extracellular matrix (ECM). Biochemical reactions with ROS result in CxL formation, which enhance corneal mechanical properties, and adjust corneal curvature.

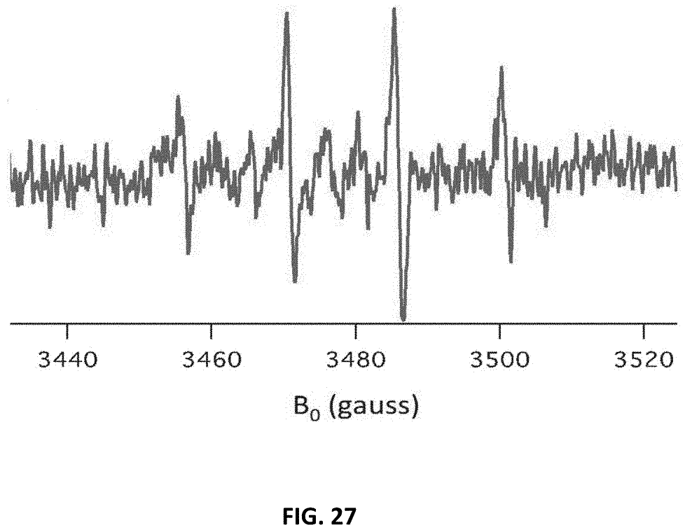

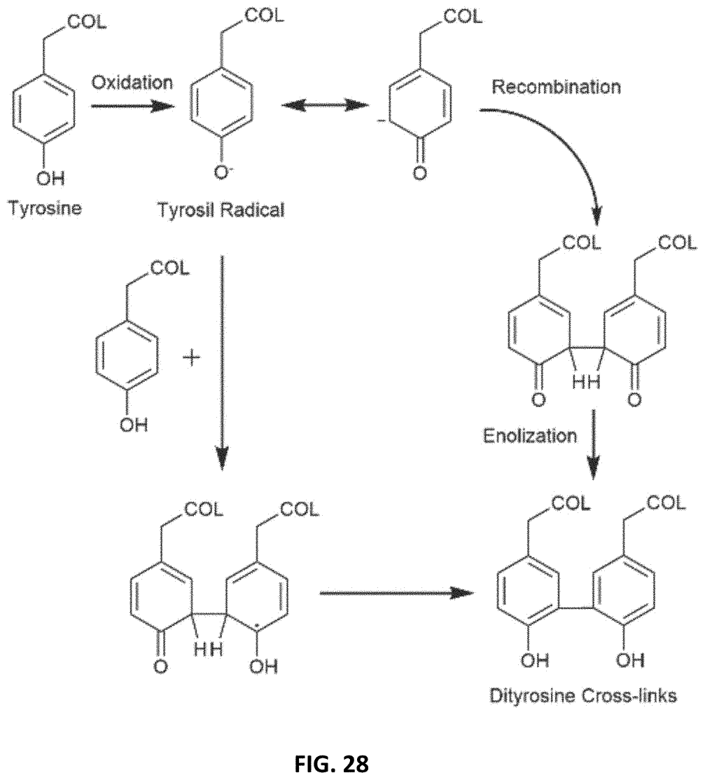

The techniques described herein incorporate a laser treatment regime such that optical breakdown never occurs. Corneal stroma mainly consists of highly organized type I collagen. When ultrashort pulses carrying nano-joule (nJ) energy are relatively loosely focused onto collagen-rich biological media, the interaction results in the formation of LDP within the focal volume and its immediate vicinity. In exemplary embodiments, LDP is used to generate an ionization field in biological media, without producing tissue-damaging thermoacoustic and shock waves. The ionization field locally ionizes and dissociates interstitial water, creating ROS (FIG. 27), which interact with surrounding proteins to form CxL (FIGS. 28, 29 and 30) giving rise to spatially resolved alterations in mechanical properties.

Crosslinking with UVA/riboflavin is based on ROS-induced formation of intra- and inter-molecular covalent bonds between collagen fibrils. Formation of free radicals through 2-photon ionization and dissociation of water molecules has initially been achieved by irradiation with high-power UV picosecond lasers. Advances in femtosecond lasers have enabled phasing to multiphoton ionization (MPI). In aqueous environments, laser-induced ionization and dissociation occur as a cascade of reactions that can be classified as primary, secondary and tertiary. Primary reactions include the formation of solvated electrons and the cation radical of water, H₂O⁺. The latter is unstable and reacts with a water molecule producing a hydrogen ion H₃O⁺, and hydroxyl radical OH*. Concurrently, dissociation of the excited water molecule occurs, H₂O*→H+OH*, providing another OH*. Primary reactions are followed by secondary and tertiary reactions in which the formation of O₂⁻, OH⁻, H₂, H₂O₂, HO₂ and other species occur.

Laser intensities focused on ROS generation have been well above the irradiance threshold for femtosecond breakdown in aqueous and ocular media (˜10¹³ W/cm2), a level at which density of photoionization-formed free electrons reach a critical value, resulting in formation of a dense, optically opaque plasma. However, since the number of free electrons produced during a single pulse is a function of irradiance, one could focus pulses generated by a femtosecond oscillator on biological media such that the density of the laser-generated free electrons is well below the critical value needed for the formation of dense plasma, but significant enough for the generation of LDP. In such a scenario, although the femtosecond irradiation is below the energy level required for optical breakdown, ionization of atoms within the focal volume is possible because the ionization probability has a number of resonance maxima due to intermediate transition of the atom to an excited state. In the vicinity of such a maximum, the ionization cross-section increases by several orders of magnitude, enabling ionization even if the frequency of the incoming electromagnetic wave is lower than the ionization potential. Multiple photons interact simultaneously with a bound electron to overcome the band gap, and produce an electron-hole pair.

Under these conditions, ionization of aqueous media is considered possible, such that LDP produces ROS in the aqueous solutions, as confirmed by the use of Electron Paramagnetic Resonance (EPR) spectroscopy to capture OH* in aqueous solution treated by a femtosecond oscillator. FIG. 27 illustrates laser-tissue interaction mechanism in cornea. FIG. 27 illustrates Electron Paramagnetic Resonance (EPR) spectrum: Spin-trap reagent 5,5-dimethy-1-pyrroline-N-oxide (DMPO) solved in Dulbecco's phosphate-buffered saline (DPBS) has trapped OH* and O₂—, created after the solution was ionized with femtosecond oscillator.

ROS initiate photo-oxidation of proteins, which results in the formation of chemical CxLs. All amino acids are susceptible to modification by *OH and *OH+O²⁻ (⁺O₂) radicals; however, tryptophan, tyrosine, histidine, and cysteine are particularly sensitive. Amino acids involved in CxL formation in corneal tissue include histidine, hydroxylysine and tyrosine. Oxidative modification of tyrosine is characterized by abstraction of phenolic hydrogen atom from tyrosine residues. The tyrosyl radical is relatively long-lived and can react with another tyrosyl radical or tyrosine to form a stable, covalent carbon-carbon bond, resulting in the creation of 1,3-dityrosine. This formation is a product of protein oxidation which leads to intra- or intermolecular CxL. FIG. 28 illustrates oxidative modification of tyrosine: Specific oxidative amino acid modification associates with abstraction of the phenolic hydrogen atom from tyrosine residues—tyrosyl radical. The tyrosyl radical is combined with another one to generate a stable, covalent, carbon-carbon bond forming 1,3-dityrosine.

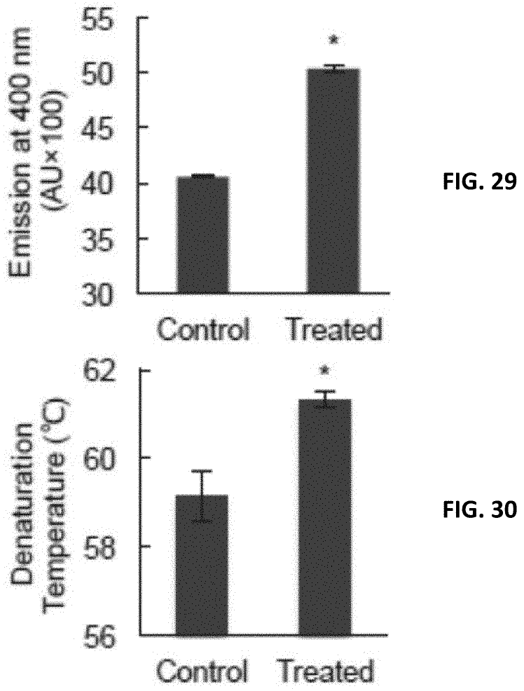

The reaction serves as a primer of pathways that lead to CxL of extracellular matrix (ECM) upon irradiation with a femtosecond oscillator. FIG. 29 illustrates the fluorescence spectrum of laser-treated and control samples of 5 mM tyrosine solution in pH 10 Tris buffer measured at 400 nm emission and 325 nm excitation. FIG. 30 illustrates differential Scanning calorimetry: The thermal denaturation temperature of treated samples is ˜2° C. higher than in controls. *p<0.05.

Several studies have been performed by laser treating porcine and rabbit corneas both, ex vivo and in vivo, that have produced highly innovative outcomes in support of the proposed aims of this application.

STUDY 1: In S1. isolated porcine corneas were tested ex vivo in an experimental design to demonstrate that the proposed approach indeed produces stiffening. Isolated porcine corneas were irradiated with the femtosecond laser, and then subjected to the inflation test to assess changes in mechanical properties. Corneas with a ring of sclera were harvested from previously frozen eyes. The scleral rim was mounted onto the custom designed supporting metal ring and fixed with cyanoacrylate glue, such that the cornea was placed at the center and left exposed. Subsequently, the upper surface of cornea was moistened with phosphate buffered saline (PBS) solution and a coverslip was placed on top of the specimen to applanate the cornea and ensure even volumetric exposure to the laser. The metal ring with attached cornea was fixed onto a 3-axis motorized translation stage (PT1, Z825B, Thorlabs, Newton, N.J.). The laser beam from femtosecond laser (HiQ IC-100 fs Nd:Glass, Spectra-Physics, Santa Clara, Calif.) was used. The pulse duration is about 80-90 femtoseconds (nominally 99 fs), the repetition rate is 52 MHz, wavelength 1060 nm, and average power after focusing objective about 60 mW. The beam was focused with Zeiss Plan-Neofluar 40×/0.6 objective, however the beam was not expanded before the back aperture of the focusing objective, and thus the NA was probably around 0.97. This allowed such large distance between consecutive treatment layers (50 microns). The beam was focused in the interior of cornea, and the motion system moved such that a planar zigzag patterns were created with 50 micron pitch at feed rate of 2.2 mm/s. Multiple planes parallel to the corneal surface were treated with 50 micron distance between two consecutive planes. Each laser irradiated plane was labeled as a “treatment layer”. A half of the cornea was laser treated. After the laser treatment, the changes in the mechanical properties were assessed via inflation test, which consisted of a series of linear load-unload regimens.

Corneal inflation test is used to assess the mechanical properties following protocols. Corneas harvested together with a scleral rim are mounted on the metal ring, with cyanoacrylate glue, with the center of the corneas left exposed. Corneas are subject to a series or linear load-unload regimens, with baseline pressure being 0.5 kPa and the maximum pressure 4 kPa. The first three cycles of loading rate 0.13 kPa/sec are utilized for pre-conditioning as a standard for comparison across different samples. The pre-conditioning cycles are also compared to post-conditioning cycle to substantiate that the sample was not degrading during the test. Two middle cycles will have a loading rate of 0.00734 kPa/sec and 0.69316 kPa/sec, respectively. Time and spatially resolved displacement maps are obtained through a two-camera digital imaging correlation (DIC) system, and the recorded maps analyzed with a commercially available software (VicSNAP and VIC3D, Correlated Solutions, Inc.)

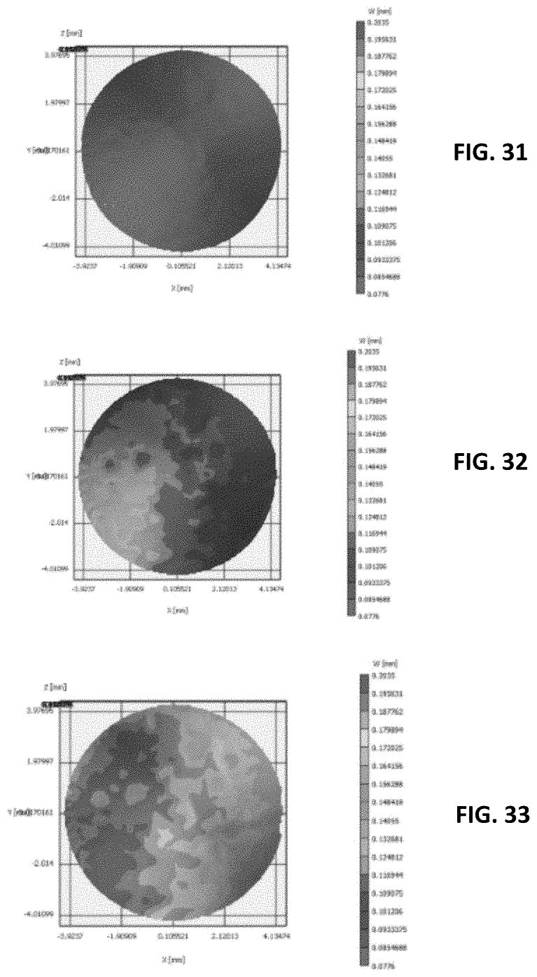

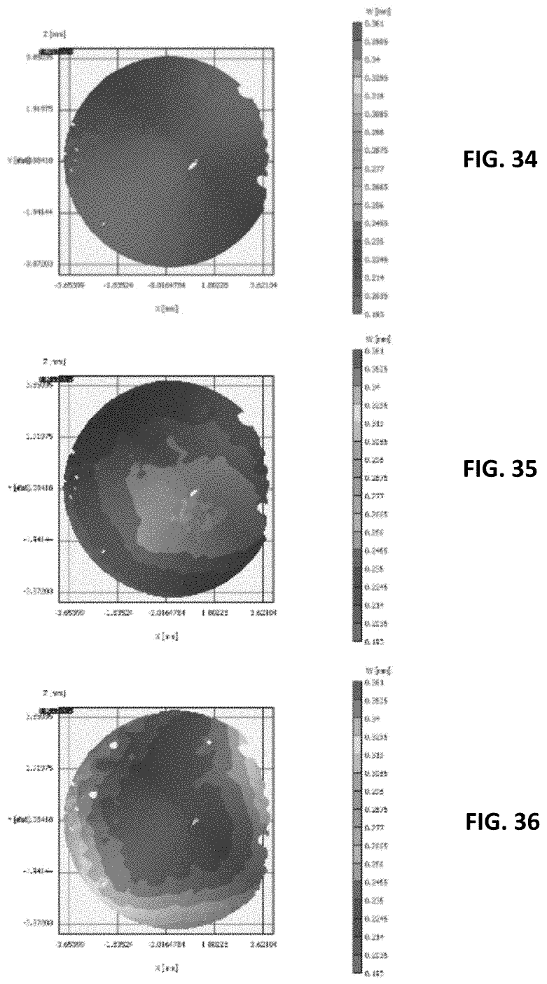

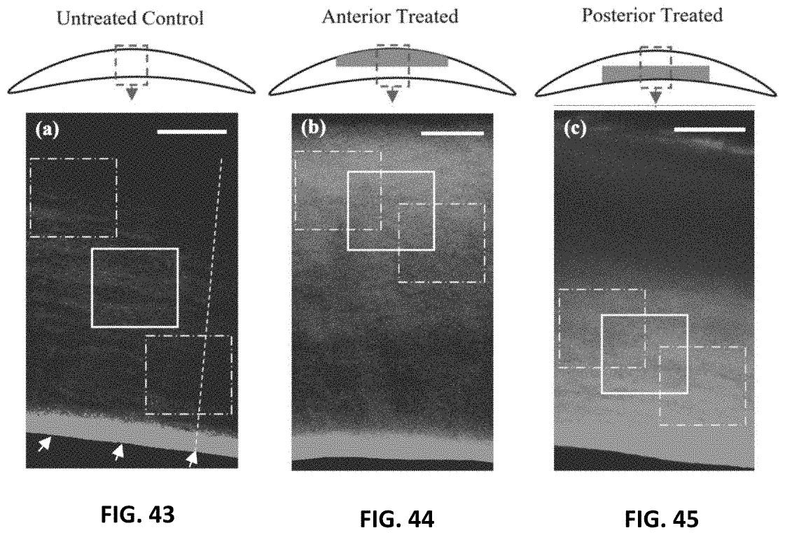

The results show significant difference in localized displacement between treated region of the cornea (2.0 kPa FIG. 31; 3.4 kPa FIG. 32; and 5.4 kPa FIG. 33) and untreated region of the cornea, whereas, control corneas exhibit anticipated symmetric displacement (2.1 kPa FIG. 34; 3.4 kPa FIG. 35; and 5.5 kPa FIG. 36). These results, together with differential scanning calorimetry (FIG. 30) and two-photon microscopy (FIGS. 43, 44 and 45) demonstrate that the laser irradiation results in CxL formation, which stiffens the cornea.

STUDY 2: A further preliminary study (S2) evaluated whether adjustment of the laser irradiation dosage was correlated with the induced CxL density, such that it is possible to control the degree of stiffening of treated corneas. The experimental design was single-factorial: Factor 1 tested the effect of laser treatment at given pulse energy (0, 2 and 5 laser ‘treatment layers’ in the interior of the cornea, with 0 representing the untreated control group).

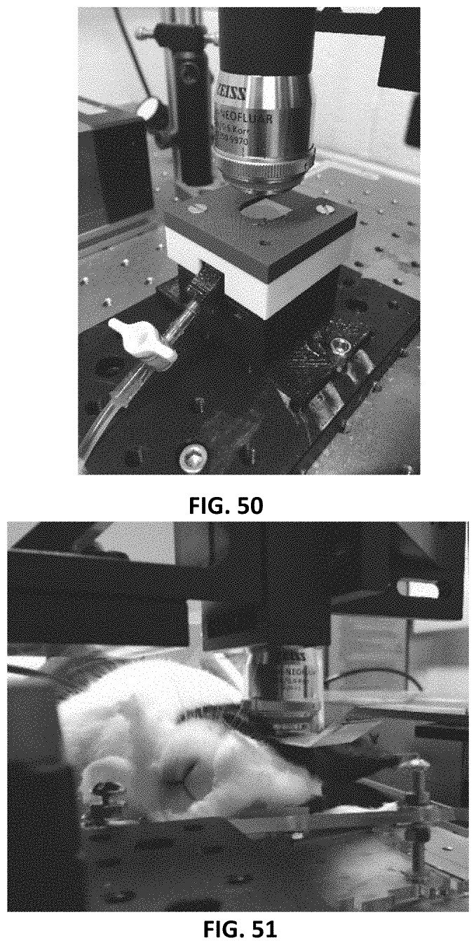

Fresh porcine eyes obtained from a local abattoir (Green Village Packing Co., Green Village, N.J.) were rinsed with 0.9% sodium chloride, inspected for presence of defects and gradually brought to a room temperature in a humidified chamber. Eyes were then mounted onto a custom-built holder and attached to an intravenous (IV) system to control the pressure (FIGS. 50-51). In-house built system centered around Ti: Sapphire tunable femtosecond laser (Coherent, Chameleon Ultra II, Santa Clara, Calif.) coupled with a high speed motion system (H-840.D2A Motion Hexapod, PI, Auburn, Mass.) was used.

A total of 21 eyes were used in the study. The eyes were divided in 3 groups. Group 1 consisted of seven eyes (n=7) and was used as a control, whereas groups 2 (n=7), and 3 (n=7), were exposed to the laser irradiation. The lasing pattern followed that described in S1, however, only the 5×5 mm region in the center of the cornea was exposed to the laser. Group 2 received two ‘treatment layers’ and group 3 has been subjected to five ‘treatment layers’. After the laser irradiation, the globe of the porcine eye was dissected, and cornea with a ring of sclera removed. Subsequent procedure followed the characterization protocol described in S1.

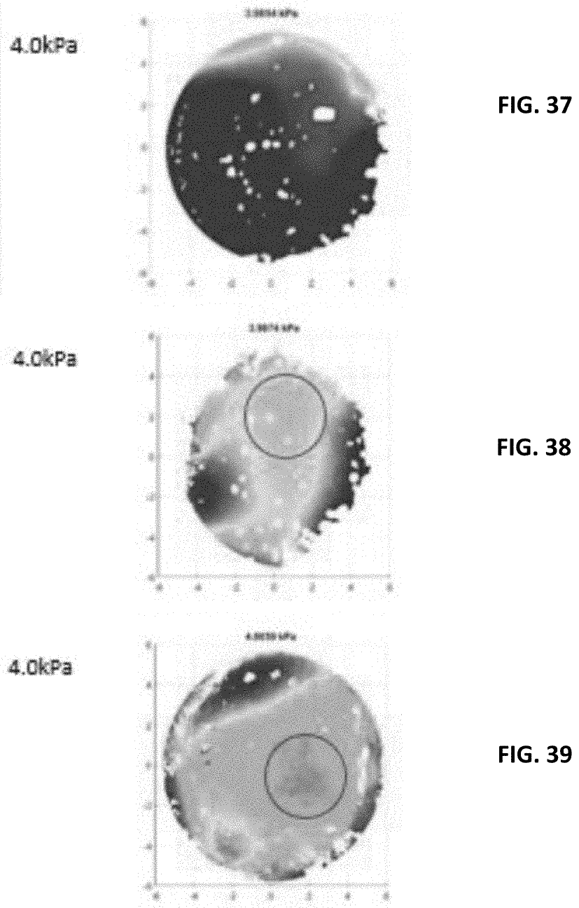

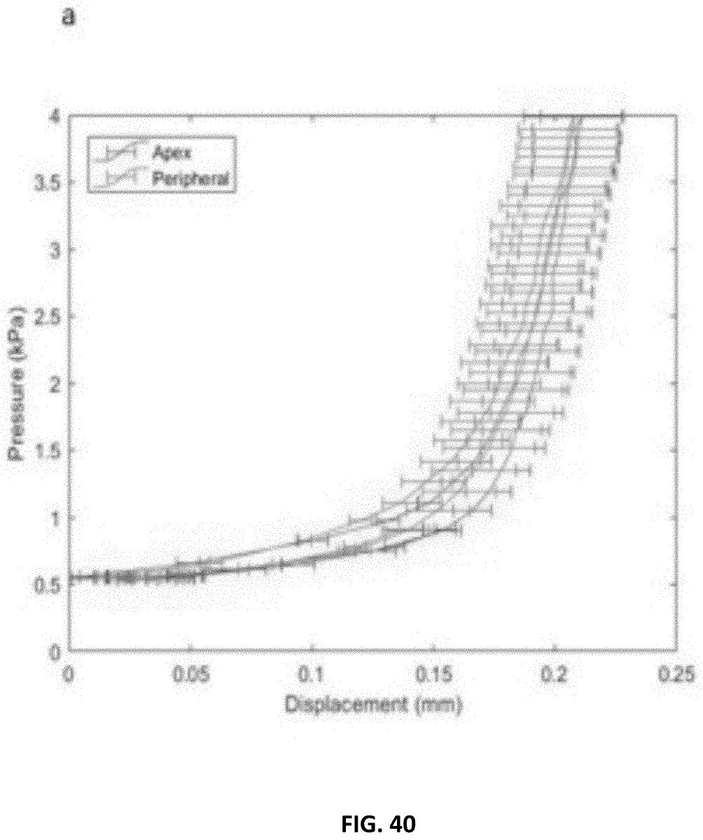

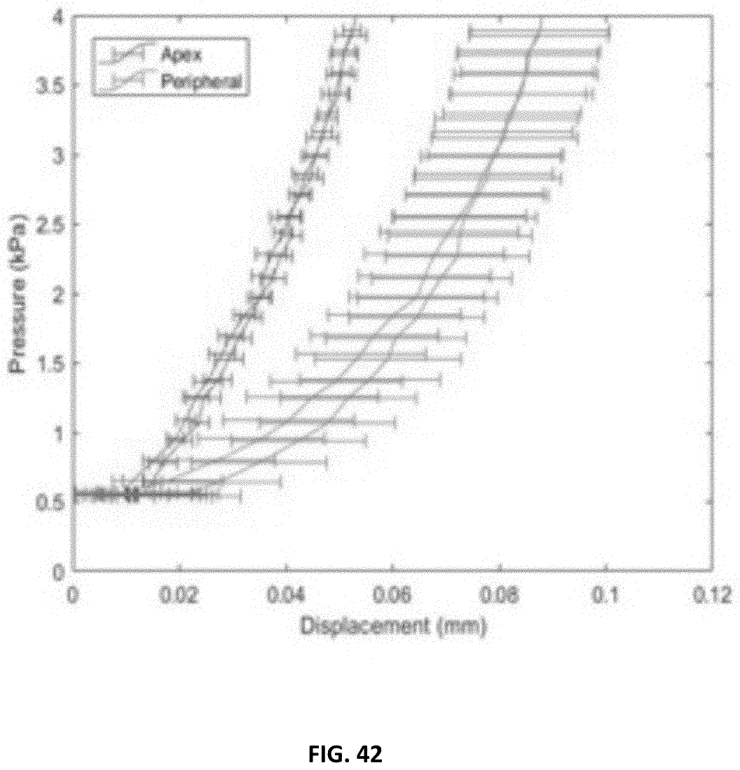

Time and spatially resolved displacement maps were obtained analogously to 51. Difference in stiffening between apex (treated) and peripheral (control) region was compared by examining displacement maps at 4.0 kPa (control FIG. 37, two treatments layers FIG. 38; and five treatment layers FIG. 39) The laser treated region is circled in FIGS. 38-39. Ten random points are extracted from each region and their hysteresis curves (FIGS. 40, 41 and 42) are compared. The difference between hysteresis curves from apex (treated) and peripheral (control) region is rather small in the control cornea (FIG. 40), and grows with the increase of the total laser irradiation (two treatments layers FIG. 41; and five treatment layers FIG. 42). Average displacement difference (in percentages) between the treated apex and non-treated peripheral region at 4 kPa (maximum pressure) 1.283±0.716 for controls, 28.257±0.716 for corneas with two laser treatment layers applied, and 93.47±11.163 for corneas subjected to five laser treatment layers.

These preliminary results demonstrated that increased exposure to the laser irradiation increases the stiffness of the stroma, suggesting that the proposed treatment modality can be tailored to provide personalized treatment.

STUDY 3: In addition to examining stiffening of the cornea, the depth dependent efficacy of crosslinking was evaluated (S3). UVA/riboflavin CxL is mostly confined to the anterior of segment of the cornea. Because the production of ROS is different than in UVA/riboflavin CxL, and due to known nonlinear absorption properties of ultrafast lasers, S3 evaluated that the proposed treatment can induce CxL at any stromal depth.

As illustrated in FIGS. 43, 44, 45 and 46, porcine and rabbit corneas were subjected to the laser treatment protocol outlined in S1 and S2. However, anterior, and posterior segment of the rabbit corneas were treated in separate experiments. In the first experiment, the laser beam was initially focused on the surface coincident to the top surface of the applanated cornea. Then five laser irradiated ‘treatment planes’ (see S1 above), were applied into cornea starting from 50 μm below the corneal surface. In the second experiment, lasing of the posterior segment was achieved by initially focusing the laser in the central cornea, and then placing subsequent treatment layers towards the posterior cornea.

After the laser irradiation, the corneas were harvested from eyes and prepared for two photon fluorescence (TPF) microscopy. Control and laser treated corneal samples were cut from isolated corneas into 2 mm² blocks with a custom-built slicer and mounted with 50% glycerol solution to a Petri dish. TPF microscope was used to obtain results depicted in FIG. 43 (untreated control), FIG. 44 (anterior laser treated) and FIG. 45 (posterior laser treated).

Corneal samples for PLM are sectioned without fixation and imaged on an Olympus IX70 inverted microscope, with AmScope MU1003 camera. PLM may be used to detect potential, laser induced, damage or changes in the refractive index of the cornea. Relative CxL density is evaluated by semi-quantitative analysis of images obtained with TPF microscopy. TPF imaging is performed with a microscope (Bruker) equipped with a tunable Mai Tai Deep See Ti: Sapphire laser (Spectra Physics) as excitation source. A 10×/0.6 NA water immersion objective (Olympus) is used to collect fluorescence signals registered with two different photomultiplier tubes, red (580-620 nm) and green (480-570 nm) wavelength regime. The excitation wavelength will be set to 826 nm, known to excite collagen matrix.

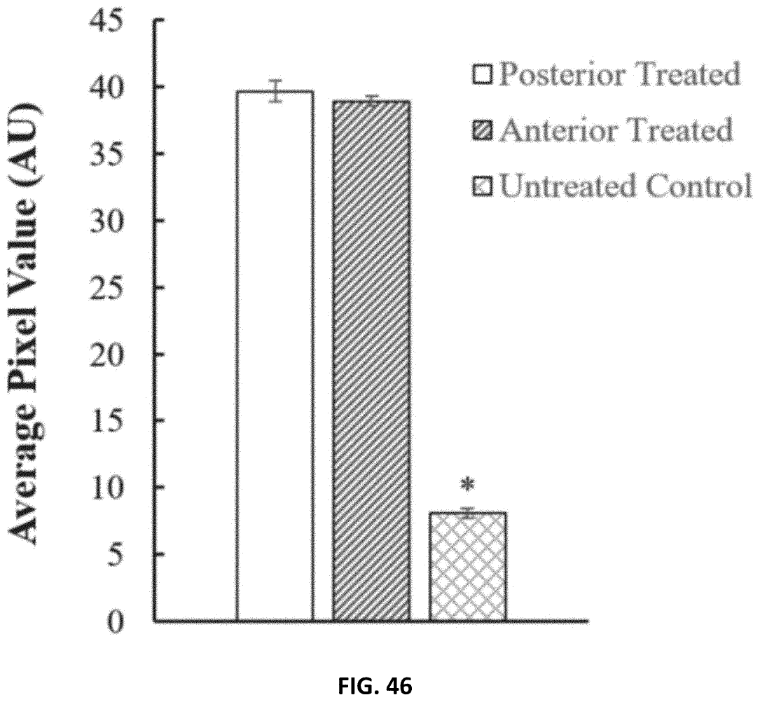

TPF images of treated anterior stroma and posterior stroma, as well as untreated control is shown in FIGS. 43-45. It is likely that the excitation of tyrosine, dityrosine oxidation products and pyridinium-type fluorophores are responsible for the contrast in the TPF images. There is a bright region in both control (FIG. 43) and laser-treated samples (FIGS. 44-45) at the bottom of the specimen, coming from excitation of Descemet's membrane, which is mostly comprised of different types of collagen. Treated regions (FIGS. 44-45) showed significantly stronger signal compared to the untreated regions, or control cornea (FIG. 43), which is in agreement with TPF images of rabbit corneal tissue crosslinked by riboflavin/UVA light. This indicates that the treatment increased CxLs density. The average pixel values of the histograms (FIG. 46), suggest that the crosslinking efficiency does not diminish as the focusing of the laser has been moved from anterior to posterior cornea.

Preliminary stability and safety studies of the proposed procedure has been addressed with in vivo studies on rabbit models. A total of 12 rabbits were used in the study. The animals were assigned to three groups. The animals in the first group (n=3) were euthanized 48 hours after laser treatment, to investigate the acute effects of laser irradiation. The animals in the second group (n=3) were euthanized after one week, to allow the eyes to undergo at least partial healing, if the laser had induced damage. The third group of animals (n=6) was monitored for 3 months.

Prior to the procedure, the animal corneas were subjected to macroscopic examination and slit-lamp evaluation by a veterinarian to ensure that there were no abnormalities or eye injuries. The rabbits were anaesthetized with an intramuscular injection of ketamine (3.5 mg kg-1) and xylazine (5 mg kg-1), and gently placed on their sides.

The eye facing upwards was treated, and the eye facing downwards was used as the control (FIG. 51). Proparacaine (0.5% ophthalmic solution) drops were applied to the treated eye for local anesthesia, followed by GenTeal water-based gel (Novartis, Alcon) to prevent corneal dehydration. The treatment protocol was based on our prior ex vivo studies (see S2 above). The treated eye was gently pressed with a coverslip to ensure the homogeneous volumetric application of laser pulses. The laser beam (HiQ IC-100 fs Nd:Glass, Spectra-Physics, Santa Clara, Calif.), focused via an objective with high NA (Zeiss, Plan Neofluar ×40/0.6 NA) on was delivered to the rabbit eye by mounting the objective on a custom-built 3-axis motorized motion system (PT1, Z825B Thorlabs, Newton, N.J.). Pulses were rasterized in the same fashion as in the ex vivo study (S2 and S3), except that each layer had a shape of a planar disc (Æ5 mm). A total of five ‘treatment layers’ were imparted into the rabbit eye.

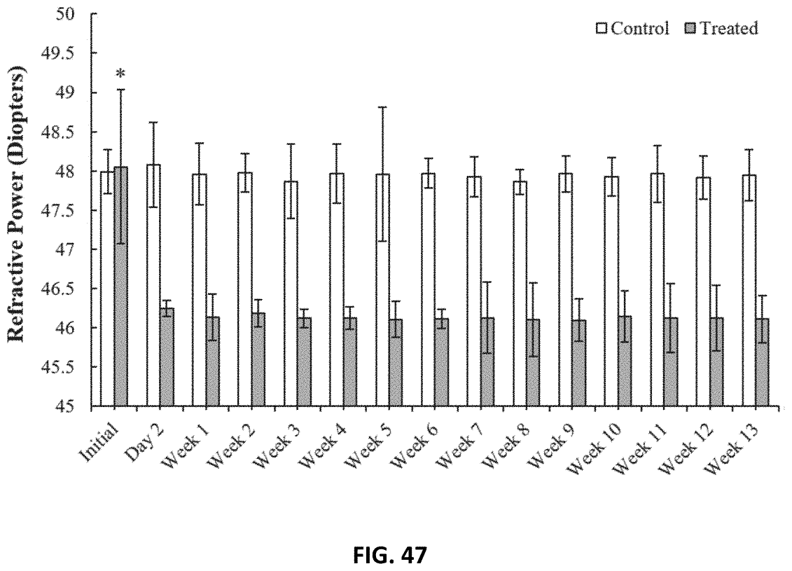

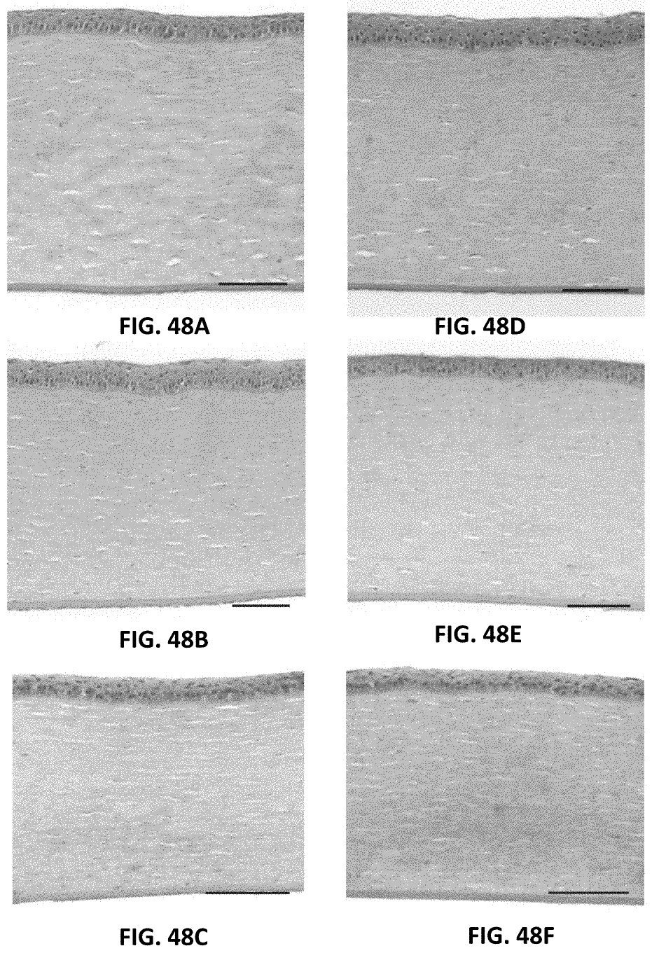

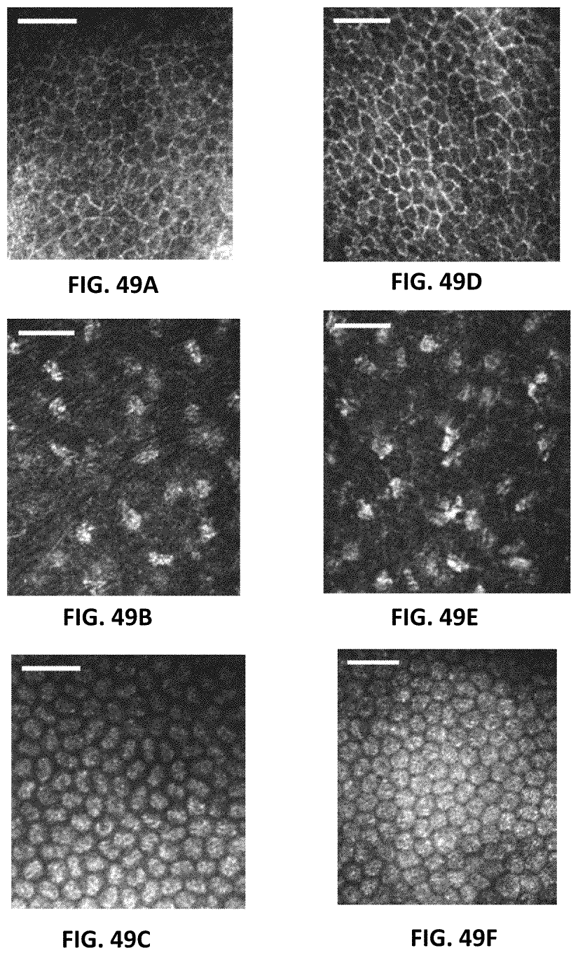

The stability of the procedure was assessed by measuring effective refractive power (ERP) of the eye and comparing it to pre-treatment value, and the control eye (FIG. 47). Safety was evaluated by examining histological sections of hematoxylin and eosin (H&E) stained corneas (FIGS. 48A-48F), as well as confocal laser scanning microscopy (CLSM) (FIGS. 49A-49F). Topography and keratometry of the eye is used as a means of assessing the treatment outcome. The corneal topography was measured with Eyesys Vision non-contact eye-topographer (EyeSys System 2000, EyeSys Vision, TX). Topographic measurements of the cornea were performed before treatment, 48 hour after treatment (Groups 1, 2 and 3), 7 days after treatment (Groups 2 and 3), and then once weekly (Group 3) (FIG. 47). Post-mortem histology (right) showed no significant damage to corneal stroma.

In Group 1, the change in ERP 48 hours after treatment was 1.74 diopters (D) relative to the pre-treatment value. In Group 2, the change in ERP 7 days after treatment was 1.64 D. The stability of the ERP changes induced by the treatment was monitored in the third group of animals. Changes remained stable 3 months after treatment, with relative difference in ERP between treated eyes and controls being about 1.94 D.

Safety of the proposed procedure. Histological staining (FIGS. 48A-48F) has revealed neither laser induced damage, nor collagen denaturation that would appear if lasing induced excessive heating of the tissue. A quantitative analysis of keratocytes and endothelial cells (FIGS. 49A-49F) with FIJI imaging software showed that cell counts were similar in treated and control eyes. Keratocyte density 48 hours after treatment was 39,464.29±2,288.57 cells per mm³ in treated eyes, and 39,523.82±5,868.68 cells per mm³ in untreated controls. Endothelial cell counts 48 hours after treatment were 2,925.00±64.14 cells per mm² in laser-irradiated corneas and 2,908.33±101.04 cells per mm² in untreated controls. Keratocyte and endothelial cell densities remained stable three months after treatment, and the counts were within the normal range.

The techniques described herein provide a treatment modality for stopping and reversing keratoconus, other ectasias, as well as to potentially offer pediatric and post-surgical therapeutic refractive error correction. The centerpiece of the proposal is a novel use ultrafast laser that allows for radically different laser-tissue (FIG. 47) Time history of treatment-induced changes in the corneal effective refractive power (ERP) of live rabbits *p<0.05: statistically significant change in refractive power. interaction. The laser-tissue interaction relies on a photochemical effect rather than optical breakdown or photoablation.

The results have confirmed the initial hypothesis that femtosecond laser can indeed produce photochemical reaction in the cornea when lasing regime is confined below the optical breakdown. It has also been shown that under the chosen set of parameters, corneal mechanical properties can be enhanced and eye refractive power amended.

If laser irradiance is about 10¹³W×cm⁻², an optical breakdown occurs, characterized by a shock wave that disrupts the surrounding tissue by creating a cavitation bubble. This process is utilized for corneal flap formation in refractive surgeries. Even if the optical breakdown is not reached, the lasing may create thermoacoustic waves that can induce significant heating of region surrounding focal volume and formation of a transient bubble, which damages and denatures the tissue. However, careful regulation of the laser parameters paired with an appropriate numerical aperture (NA), confines the free electron density below a critical value (˜10²¹ cm⁻³). In such a scenario, the interaction between laser irradiation and corneal stroma is mainly photochemical. The operating envelope ranges between increased thermal effects, which denature extracellular matrix, and the insufficient ionization, in case of which the chemical reactions are either absent or do not produce desired goals. However, the ionization mode needs to be considered as well. Below the optical breakdown threshold two ionization mechanisms are possible, tunneling (TI) and multiphoton ionization (MPI). In TI the laser electric field suppresses the Coulomb well that binds the electron in valence, electron then tunnels through the barrier and becomes free. MPI occurs when an electron simultaneously absorbs several photons, whose combined energy is greater than the band gap of the material. Photoionization can be dominated by one of the two modes, or be a combination of both. It is likely that photoionization rates are higher when MPI dominates the process, however, there is a risk of substantial heating. On the other hand, TI can trigger resonance between the incident electromagnetic filed and the electronic transition. In that case excitation of the atom occurs, followed by the ionization. TI may be possible at very low pulse energies that practically guarantee absence of any laser-based adverse effects, but the treatment could be unstable. Therefore, it is important to find optimal laser conditions that produce highest rate of the photochemical reactions needed for CxL, while minimizing adverse effects.

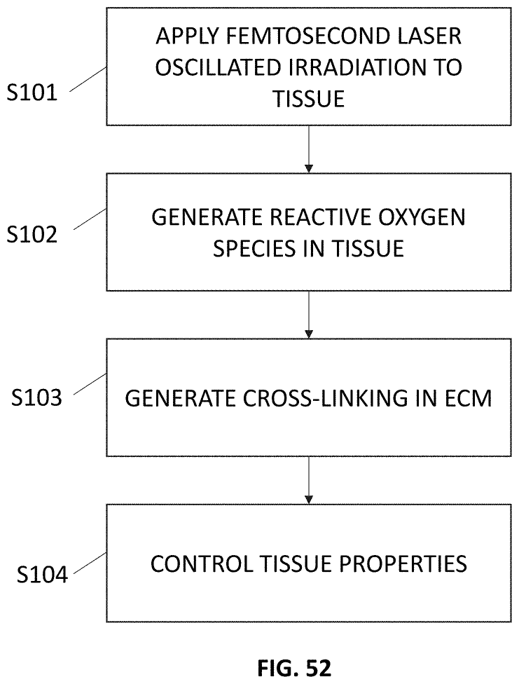

Referring now to FIG. 52, an example of a method of modulating cytokine activity within a tissue, for example a cornea is shown. As described in International Publication No. WO 2017/070637 and U.S. Patent Application Publication Nos. 2018/0193188 and 2018/0221201, tissue treatment can be achieved without the need for exogenous photosensitizers such as riboflavin by ionizing water within ophthalmologic (e.g., corneal) or other tissue to generate reactive oxygen species.

In step S101, a light source irradiates tissue. In some embodiments, the light source is a femtosecond laser oscillator. In some embodiments, the tissue is eye tissue, for example, tissue of the cornea.

In step S102, the femtosecond laser induces a low-density plasma that generates an ionization field resulting in the generation of reactive oxygen species (ROS) in and around the tissue. Treatment can be achieved over a broad range of wavelengths, e.g., including 1060 nm. The pulse duration is about 80-90 femtoseconds, the repetition rate is 52 MHz, and average power after focusing objective is between about 10 mW and about 100 mW, e.g., about 60 mW. The beam was focused with Zeiss Plan-Neofluar 40×/0.6 objective, however the beam was not expanded before the back aperture of the focusing objective, and thus the NA was probably around 0.97. The distance between consecutive treatment layers is 50 microns.

In step S103 and without being bound by theory, the light source and/or the resulting ROS interact with collagen fibrils in the extracellular matrix (ECM). Biochemical reactions with ROS result in CxL formation.

In step S104 and without being bound by theory, the light source and/or the resulting ROS controls tissue remodeling. Such tissue remodeling including changing the stiffness in the corneal tissue, resulting in changes in the optical characteristics, such as refractive power. In some embodiments, the tissue remodeling including applying a mechanical load to the tissue, e.g., the steepening or flattening procedures described herein.

As discussed herein, an ultrafast laser-tissue interaction method is disclosed. When lasing is restricted below the optical breakdown, it can ionize and dissociate interstitial water in collagenous tissues. The ionization results in production of refractive oxygen species, which in turn interact with collagen and form crosslinking. There are numerous potential applications of this process in translational medicine including ophthalmology and orthopedics. Ultrafast laser-based corneal crosslinking is as an attractive choice due to absence of photosensitizers, and no need for epithelial debriding. Crosslinking of corneal tissue can be used to treat keratoconus, and for noninvasive vision correction. The Latter application includes the combination of crosslinking, which can be done with an ultrafast laser or with more traditional methods (riboflavin/UVA light), and application of mechanical load.

Femtosecond laser-produced low density plasma results in water ionization and subsequent crosslinking. Without being bound to a particular theory, it is believed that incoming photons need to be in resonance with interstitial water in collagenous tissue to trigger ionization. There may be more than one resonance peak, and one of the peaks may be Fano resonance. Further, the laser pulse duration is below 100 femtoseconds to produce the resonance.

A femtosecond laser-based crosslinking system as disclosed herein is capable of treating an eye within a clinically relevant timeframe, without sacrificing accuracy and efficiency. Laser pulses can be delivered via free-space or fiber-optics, and spatially resolved treatment realized with a high speed, computer controlled motion system. Crosslinking itself increases corneal rigidity and thus can retard progression of keratoconus. On the other hand when crosslinking is paired with carefully tuned mechanical loading, it can reshape corneal curvature, which is used for correction of refractive errors.

Cytokine Modulation: Femtosecond laser treatment of corneal stroma disclosed herein prevents cytokine induced appoptosis of corneal keratocytes. This technique is particularly useful in managing inflammation.

The beneficial effects of the cytokine oxidation towards inflammation management can be introduced pharmacologically as well. This approach may have broader spectrum of applications beyond tissues that can be reached with a femtosecond laser.

Articular Cartilage, meniscus, ligament, tendon and other collagen rich tissues: Experimental data have been gathered for the femtosecond laser cross-linking of articular cartilage, showing that laser cross-linking enhances (a) the compressive stiffness of healthy bovine and arthritic human cartilage, and (b) the wear resistance of healthy bovine cartilage. These experiments have provided specific laser settings that successfully achieve these results.

A prototype of a probe that transmits the laser beam through a fiber-optic cable has been developed and shown to successfully reproduce the cross-linking of collagen, with equivalent or better results than the tabletop set up. This fiber-optic probe can be used to treat collagenous tissues (such as cartilage, ligament, tendon, meniscus) arthroscopically.

Using laser crosslinking to “glue” collagenous tissues together across an interface, by creating crosslinks between collagen molecules/fibrils/fibers located on both sides of the interface. Adding a collagen-based liquid or gel at the interface between collagenous tissues and using laser crosslinking to increase crosslinking sites across the interface, thus enhancing the “gluing” mechanism.

This technology can have widespread applications in the treatment of collagenous tissues, including (but not limited to): (a) repairing meniscal tears in the tibiofemoral and temporomandibular joints; (b) gluing cartilage surfaces in osteochondral repairs that use allografts or autografts (e.g., “mosaicplasty”); (c) repairing ligament tears; (d) repairing tendon tears; (e) repairing skin cuts and tears (plastic surgery).

It is understood that the subject matter described herein is not limited to particular embodiments described, as such may, of course, vary. It is also understood that the terminology used herein is for the purpose of describing particular embodiments only, and is not intended to be limiting. Where a range of values is provided, it is understood that each intervening value between the upper and lower limit of that range and any other stated or intervening value in that stated range, is encompassed within the disclosed subject matter.

What is claimed is:

1. A method of treatment of the cornea of an eye comprising:
exposing the cornea to a crosslinking medium, and
simultaneously applying a mechanical loading to the cornea, wherein the mechanical loading is selected as a strain proportional to a dimension of the eye.

2. The method of claim 1, wherein the mechanical loading is a 15% deformation (measured by a micrometer) of the original eye ball height.

3. The method of claim 1, wherein the mechanical loading is provided by a 1.6 mm diameter cylinder stick.

4. The method of claim 1, wherein the crosslinking medium comprises at least one of UV-A light and riboflavin.

5. The method of claim 1, wherein the mechanical loading is selected as a portion of the cornea.

6. A method of treatment of the cornea of an eye comprising:
applying mechanical loading to the cornea; and
irradiating a central region of the cornea with a femtosecond laser while the cornea is deformed by the mechanical loading.

7. The method of claim 6, wherein the mechanical loading is achieved by pressing the cornea with a cover slip.

8. A method of treatment of the cornea of an eye comprising:
applying mechanical loading to the cornea; and
irradiating a central region of the cornea with a steepening technique with a femtosecond laser while the cornea is deformed by the mechanical loading, wherein the laser irradiation is applied in an annulus shaped pattern, such that apical region of cornea remains intact while the peripheral region is irradiated with the laser.

9. A method of altering the curvature of the cornea comprising:
controlling a light source to apply light energy pulses to corneal tissue; wherein the light energy pulses are below an optical breakdown threshold for the cornea; ionize water molecules within the treated corneal layer to generate reactive oxygen species; and initiate crosslinking within the extracellular matrix of the cornea to change the stiffness of the cornea.

10. The method of claim 9, wherein the light source is a laser.

11. The method of claim 10, wherein the laser is a femtosecond laser.

12. The method of claim 9, wherein the light energy pulses have an average power output between about 10 mW and about 100 mW.

13. The method of claim 12, wherein the light energy pulses have an average power output of about 60 mW.

14. The method of claim 9, wherein the light energy pulses have a pulse energy between about 0.9 nJ and about 1.5 nJ.

15. The method of claim 9, wherein the light energy pulses have a pulse energy of about 1.2 nJ.

16. The method of claim 9, wherein the light energy pulses have a wavelength of about 1060 nm.

17. The method of claim 9, wherein controlling the light source comprising applying the light energy pulses in one or more layers to the tissue.

18. The method of claim 17, wherein the one or more layers are spaced about 50 microns apart.

19. The method of claim 17, wherein controlling the light source comprising applying the light energy pulses in two to five layers to the tissue

20. The method of claim 9, further comprising applying a mechanical loading to the cornea.
